(19) United States
(12) Patent Application Publication
(10) Pub. No.: US 2023/0031369 A1
(43) Pub. Date: Feb. 2, 2023

(54) THERAPEUTIC INTERACTIONS OF LEUCOMETHYLTHIONINIUM

(71) Applicant: WisTa Laboratories Ltd., Singapore (SG)

(72) Inventors: Gernot Riedel, Aberdeen, Aberdeenshire (GB); Valeria Melis, Aberdeen, Aberdeenshire (GB); Charles Robert Harrington, Aberdeen, Aberdeenshire (GB); Jochen Klein, Frankfurt (DE); Grazyna Niewiadomska, Warsaw (PL); Marta Steczkowska, Warsaw (PL); Karima Schwab, Berlin (DE); Claude Michel Wischil, Aberdeen, Aberdeenshire (GB)

(73) Assignee: WisTa Laboratories Ltd., Singapore (SG)

(21) Appl. No.: 17/620,949

(22) PCT Filed: Jun. 30, 2020
(86) PCT No.: PCT/EP2020/068422

§ 371 Date: Dec. 20, 2021

(30) Foreign Application Priority Data
Jul. 1, 2019 (GB) 1909493.7

(51) Int. Cl. A61K 31/5415; A61P 25/28; A61K 45/06

(52) U.S. Cl. CPC A61K 31/5415; A61P 25/28; A61K 45/06

(57) ABSTRACT

The present invention relates generally to methods of treatment of Alzheimer's Disease or Mild Cognitive Impairment which are adapted to avoid negative interactions between combinations of therapeutics. In particular there are disclosed such methods of treatment in which the order of therapeutics is actively controlled to mitigate homeostatic downregulation prior to administration of active, for example disease modifying, therapeutic agents. In certain embodiments therapy with symptomatic treatments (such as modifiers of the activity of acetylcholine or glutamate neurotransmitters) may subsequently be combined with the disease modifying or other active treatment. The invention also applies the findings in relation to homeostatic downregulation to novel methods of clinical trial design.

### CROSS-REFERENCE TO RELATED APPLICATIONS

This application is a National Stage filing under 35 U.S.C. 371 of International PCT Application No. PCT/EP2020/068422, filed Jun. 30, 2020, which claims priority to Great Britain Application No. 1909493.7, filed Jul. 1, 2019. The contents of these applications are incorporated herein by reference in their entirety.

### TECHNICAL FIELD

The present invention relates generally to methods of treatment of Alzheimer's Disease or Mild Cognitive Impairment which are adapted to avoid negative interactions between combinations of therapeutics, or to enhance the effect of therapeutics.

### BACKGROUND ART

The only treatments currently available for the treatment of Alzheimer's disease (AD) are symptomatic. The most widely used of these are the acetylcholinesterase inhibitors (AChEIs) which work by chronically increasing the levels of acetylcholine (ACh) in the synaptic cleft. In experimental models, cholinergic function is associated primarily with selective attention (Botly and De Rosa, 2007; 2008; Sarter et al., 2016), and is not particularly sensitive to more broadly based measures of functional impairment/improvement (reviewed in (Klinkenberg and Blokland, 2010; Robinson et al., 2011)). Similar considerations apply to memantine which also modulates brain function in a non-specific manner (Gastambide et al., 2012; Ding et al., 2018). The therapeutic benefits of these treatments are relatively short-lasting (Courtney et al., 2004), with fewer than 30% of patients continuing on AChEIs 12 months after initiation (Mauskopf et al., 2005; Singh et al., 2005; Raina et al., 2008). A substantial proportion of AD patients are in any case not treated, ˜44% in the US (Koller et al., 2016) and ˜77% in UK (Martinez et al., 2013). In France, reimbursement for these drugs has been withdrawn because of “insufficient medical benefit and dangerousness because of side effects” (Krolak-Salmon et al., 2018). The low medical use and patient adherence are due to low perceived efficacy and side effects, with rate of decline after temporary symptomatic improvement no different from that occurring in the absence of treatment (Courtney et al., 2004). Hence it is agreed generally that a major unmet medical need exists to develop a treatment able to slow the progression of AD. A Lancet Neurology Commission report (Winblad et al., 2016) noted that “ . . . no treatment is yet available to halt or reverse the underlying pathology of established AD. Indeed, an effective therapy for AD is perhaps the greatest unmet need facing modern medicine.” The last new treatment to have been approved for AD was in 2003 for memantine (Lanctôt et al., 2009). From 2002 to 2012, there have been 289 clinical trials at Phase 2 or Phase 3, with an overall failure rate of 99.6% (Cummings et al., 2014). There have been 19 further trial failures since 2012 targeting various aspects of pathological processing of β-amyloid (Panza et al., 2019).

Despite the limitations of available symptomatic treatments, almost all late-stage clinical trials currently ongoing or recently completed aiming to test new therapeutic approaches have been conducted in patient populations where the majority of subjects continue taking symptomatic treatments (Panza et al., 2019). This is determined, in part, by ethical concerns that patients participating in potentially lengthy clinical trials would be denied access to any treatment if randomised to the placebo arm. A further consideration has been the unproven assumption that symptomatic treatments do not interfere with treatments targeting the underlying pathology because their modes of action are different.

Methylthioninium (MT) acts as a tau aggregation inhibitor in vitro (Wischik et al., 1996; Harrington et al., 2015), dissolves PHFs from Alzheimer's disease brain tissue, (Wischik et al., 1996) and reduces tau pathology and associated behavioural deficits in transgenic mouse tau models at brain concentrations consistent with human oral dosing (Melis et al., 2015a; Baddeley et al., 2015).

Leuco-methylthioninium bis(hydromethanesulfonate) (LMTM; USAN name hydromethylthionine mesylate) is being developed as a treatment targeting pathological aggregation of tau protein in AD (Wischik et al., 2018). The methylthioninium (MT) moiety can exist in oxidised (MT⁺) and reduced (LMT) forms. LMTM is a stabilised salt of LMT which has much better pharmaceutical properties than the oxidised MT⁺ form (Baddeley et al., 2015; Harrington et al., 2015). We have reported recently that LMT rather than MT⁺ is the active species blocking tau aggregation in vitro (Al-Hilaly et al., 2018). LMT blocks tau aggregation in vitro in cell-free and cell-based assays (Harrington et al., 2015; Al-Hilaly et al., 2018), and reduces tau aggregation pathology and associated behavioural deficits in tau transgenic mouse models in vivo at clinically relevant doses (Melis et al., 2015a). LMT also disaggregates the tau protein of the paired helical filaments (PHFs) isolated from AD brain tissues converting the tau into a form which becomes susceptible to proteases (Wischik et al., 1996; Harrington et al., 2015).

The MT moiety also has a range of other potentially beneficial properties. It has been known for some time that at low concentrations (10-100 nM) it enhances mitochondrial activity by acting as a supplementary electron carrier in the electron transfer chain. The MT moiety undergoes redox cycling catalysed by complex I using NADH as co-factor whereby it accepts electrons which are subsequently transferred to complex IV (Atamna et al., 2008; Atamna et al., 2012). It is able also to induce mitochondrial biogenesis and to activate Nrf2-mediated oxidative stress response elements in vivo (Stack et al., 2014). Other activities include neuroprotective effects in the brain by inhibiting microglial activation and increasing autophagy (Zhao et al., 2016). The MT moiety has been shown to increase the clearance of pathological tau in vivo via enhancement of autophagy at the 10-20 nM concentration range (Congdon et al., 2012). Therefore, in addition to dissolution of AD tau aggregates, LMTM has numerous complementary actions that address many of the pathways currently advocated as having potential for the treatment of AD (Oz et al., 2009; Schirmer et al., 2011).

Although LMTM given orally produces brain levels sufficient for activity in vitro and in vivo (Baddeley et al., 2015), it had minimal apparent efficacy if taken as an add-on to symptomatic treatments in two large Phase 3 clinical trials (Gauthier et al., 2016; Wilcock et al., 2018). In subjects receiving LMTM as monotherapy, however, treatment produced marked slowing of cognitive and functional decline, reduction in rate of progression of brain atrophy measured by MRI and reduction in loss of glucose uptake measured by FDG-PET (Gauthier et al., 2016; Wilcock et al., 2018). When these outcomes were analysed in combination with population pharmacokinetic data available from subjects participating in the trials, LMTM was found to produce concentration-dependent effects whether taken alone or in combination with symptomatic treatments. However, the treatment effects in monotherapy subjects were substantially larger than in those taking LMTM in combination with symptomatic treatments.

WO2008/155533 concerns MT containing compounds for treating MCI.

WO2009/060191 concerns the design of clinical trials for putative therapeutics for neurodegenerative disorders. It is specifically envisaged that subjects on active symptomatic treatment can be included in the trials.

WO2018/019823 describes novel regimens for treatment of neurodegenerative disorders utilising methylthioninium (MT)-containing compounds. That publication summarises earlier disclosures of MT containing compounds, and particularly “LMTX” compounds for treating such disorders including AD and Mild Cognitive Impairment (MCI). WO2018/019823 identified two key factors. The first was in relation to the dosage of MT compounds, and the second was their interaction with symptomatic treatments.

WO2020/020751 describes novel dosing regimens for LMT compounds which maximise the proportion of subjects in which the in vivo MT concentration will exceed that required to demonstrate therapeutic efficacy.

### DISCLOSURE OF THE INVENTION

The present studies were undertaken with the aim of understanding the mechanisms responsible for the reduced efficacy of LMTM as an add-on to symptomatic treatments discussed above. In these studies a well-characterised tau transgenic mouse model (Line 1, “L1”; (Melis et al., 2015b)) was compared with wild-type mice.

We demonstrated that in tau transgenic mice expressing the short tau fragment which constitutes the tangle filaments of AD, LMTM given alone increased hippocampal acetylcholine (ACh) levels, glutamate release from synaptosomal preparations, synaptophysin levels in multiple brain regions and mitochondrial complex IV activity, reduced tau pathology, restored choline acetyltransferase (ChAT) immunoreactivity in basal forebrain and reversed deficits in spatial learning.

Chronic pretreatment with rivastigmine was found to reduce or eliminate almost all these effects, apart from reduction in tau aggregation pathology and restoration of ChAT immunoreactivity in basal forebrain. LMTM effects on hippocampal ACh and synaptophysin levels were also reduced in wild-type mice.

Thus interference with LMTM efficacy by a cholinesterase inhibitor observed clinically can also be reproduced in a tau transgenic mouse model and, to a lesser extent, in wild-type mice.

We observed that pretreatment with symptomatic drug altered a broad range of brain responses to LMTM across different transmitter systems and cellular compartments at multiple levels of brain function. There was no single locus for the negative interaction. Rather, the chronic neuronal activation induced by reducing cholinesterase function produced compensatory homeostatic downregulation in multiple neuronal systems. This has the effect of reducing a broad range of treatment responses to LMTM associated with reduction in tau aggregation pathology.

Importantly, since the interference is dictated by homeostatic responses to prior symptomatic treatment, it is logical that there would be similar interference with other drugs tested as “add-on” to existing symptomatic treatment, regardless of the intended therapeutic target or mode of action. The present overview outlines key results which now provide a working model to explain interference by symptomatic treatment. Put another way, it may be expected that any activating pretreatment will produce a homeostatic compensatory downregulatory response which will attenuate the effectiveness of the second treatment if it too is activating.

The experiments we conducted were designed to mimic the clinical situation in which LMTM is added in patients already receiving symptomatic treatments. However a further implication of the model is in relation to treatments in which LMTM (or other therapeutic) are commenced prior to the symptomatic treatments described above. If homeostatic downregulation is determined by the treatment that comes first, it is logical that the treatment effects of LMTM would dominate, albeit that the response to add-on symptomatic treatment could be reduced to some extent if introduced later as “add-on” therapy.

Additional experiments were conducted to establish the effect of withdrawal of LMTM after 18 months of treatment. In these experiments the patient group was split according to whether they has received LMTM as an add-on to existing symptomatic therapy (AChEI, Memantine “Achmem”) or as a monotherapy. It was further split according to whether subjects had received a higher Cmax exposure to MT or a lower one, according to the pharmacokinetic modelling described in WO2020/020751.

Unexpectedly, the high Cmax add-on group actually showed an unexpected improvement in ADAS-cog following LMTM withdrawal, which is consistent with a disease-modifying effect during LMTM treatment which results in an improved response to symptomatic treatment alone. This finding has potential implications for the use of LMTM and Achmem combination therapies—for example in patient groups who have proved non-responsive to Achmem, the disease modifying effects of LMTX may actually enhance the response to Achmem, particularly once the LMTX is discontinued, and particularly in relation to the ADAS-cog in mild AD subjects. This has utility since many patients do not respond to Ach inhibitor treatment (for example 30-40%, or more—see e.g. McGleenon B M, Dynan K B, Passmore A P. Acetylcholinesterase inhibitors in Alzheimer's disease. Br J Clin Pharmacol. 1999; 48(4):471-480. doi:10.1046/j.1365-2125.1999.00026.x).

Accordingly the present disclosure relates to methods of treatment of Alzheimer's disease or Mild Cognitive Impairment in which the order of therapeutics is actively controlled e.g. to prevent homeostatic downregulation prior to administration of active therapeutic agents, both in relation to MT and non-MT-containing compounds. In certain embodiments therapy with the neurotransmission modifying compounds discussed above (such as modifiers of the activity of acetylcholine or glutamate neurotransmitters) may subsequently be combined with, or follow, the MT or non-MT-containing compound treatment.

The present disclosure also utilises the present findings in relation to clinical trial design.

Thus in one aspect there is provided a method of therapeutic treatment of Alzheimer's disease or Mild Cognitive Impairment in a subject,
which method comprises administering to said subject a therapeutic methylthioninium (MT)-containing compound,
wherein the MT-containing compound is an LMTX compound of the following formula:

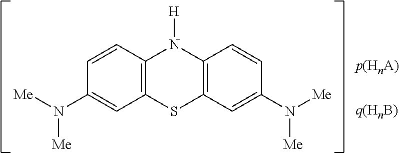

wherein each of HₙA and HₙB (where present) are protic acids which may be the same or different,
and wherein p=1 or 2; q=0 or 1; n=1 or 2; (p+q)×n=2,

or a hydrate or solvate thereof,
wherein said subject is selected from the following groups:

(i) a subject who has not historically received treatment with a neurotransmission modifying compound which is a modifier of the activity of acetylcholine or glutamate neurotransmitters; or

(ii) a subject who has historically received treatment with a neurotransmission modifying compound which is a modifier of the activity of acetylcholine or glutamate neurotransmitters, but ceased that treatment at least 3, 4, 5, 6, 7, or 8 weeks prior to treatment with the LMTX compound;
wherein said therapeutic treatment with LMTX is maintained for treatment timeframe without co-administration with a neurotransmission modifying compound which is a modifier of the activity of acetylcholine or glutamate neurotransmitters
followed by;
treatment with LMTX with co-administration of a neurotransmission modifying compound which is a modifier of the activity of acetylcholine or glutamate neurotransmitters.
wherein the treatment timeframe prior to co-administration of the therapeutic compound with the neurotransmission modifying compound is at least 2 months.

In another aspect there is provided a method of therapeutic treatment of Alzheimer's disease or Mild Cognitive Impairment in a subject,
which method comprises administering to said subject a therapeutic compound which does not contain MT,
wherein said subject is selected from the following groups:

(i) a subject who has not historically received treatment with a neurotransmission modifying compound which is a modifier of the activity of acetylcholine or glutamate neurotransmitters; or

(ii) a subject who has historically received treatment with a neurotransmission modifying compound which is a modifier of the activity of acetylcholine or glutamate neurotransmitters, but ceased that treatment at least 3, 4, 5, 6, 7, or 8 weeks prior to treatment with the LMTX compound;
wherein said therapeutic treatment with said therapeutic compound is maintained for a treatment timeframe without co-administration of a neurotransmission modifying compound which is a modifier of the activity of acetylcholine or glutamate neurotransmitters
optionally followed by;
treatment with said therapeutic compound with co-administration with a neurotransmission modifying compound which is a modifier of the activity of acetylcholine or glutamate neurotransmitters.
wherein the treatment timeframe prior to co-administration of the therapeutic compound with the neurotransmission modifying compound is at least 2 months.

In another aspect there is provided a method for assessing the efficacy of a compound which does not contain MT which is putatively therapeutic for Alzheimer's disease or Mild Cognitive Impairment in a subject, the method comprising the steps of:

(a) selecting a subject group which consists only of:
(i) subjects who have not historically received treatment with a neurotransmission modifying compound which is a modifier of the activity of acetylcholine or glutamate neurotransmitters; and
(ii) subjects who have historically received treatment with a neurotransmission modifying compound which is a modifier of the activity of acetylcholine or glutamate neurotransmitters, but ceased that treatment at least 3, 4, 5, 6, 7, or 8 weeks prior to said assessment;

(b) stratifying the subject groups into at least 2 sub-groups according to a baseline indicator of likely disease progression,

(c) treating members of each subject group with the non-MT-containing compound for a treatment timeframe,

(d) deriving psychometric and optionally physiological outcome measures for each treated patient group,

(e) comparing the outcomes at (d) with a comparator arm of said sub-groups which is optionally a placebo or minimal efficacy comparator arm,

(f) using the comparison in (e) to derive an efficacy measure for the non-MT-containing compound.

In these aspects, where there has been historical treatment, it is ceased 3, 4, 5, or 6 weeks or more prior to treatment or assessment, most preferably 6 weeks before.

In these aspects, where there is to be co-administration, the treatment timeframe prior to co-administration of the therapeutic compound with the neurotransmission modifying compound is preferably at least 2, 3, 4, 5 or 6 months, most preferably at least 6 months.

In another aspect there is provided a method of therapeutic treatment of Alzheimer's disease or Mild Cognitive Impairment in a subject,

which method comprises administering to said subject a therapeutic compound,
wherein said subject is receiving treatment with a neurotransmission modifying compound which is a modifier of the activity of acetylcholine or glutamate neurotransmitters;
wherein said therapeutic treatment with said therapeutic compound is maintained for a first treatment timeframe with co-administration of the neurotransmission modifying compound, during which treatment timeframe the dosage of said neurotransmission modifying compound is reduced to zero, followed by
treatment with said therapeutic compound without co-administration with a neurotransmission modifying compound for a second treatment timeframe,
wherein the first treatment timeframe is between 2 weeks and 8 weeks.
wherein the second treatment timeframe is at least 3 months, more preferably at least 6 months or at least 12 months.

The first treatment timeframe may more preferably be between 3 and 8 weeks, e.g. 3 to 6 weeks, or be about 1 or 2 months. Preferably the reduction in dosage is steady e.g. linear one over the period so that the subject is not exposed to sudden withdrawal.

By “titrating” the subject off the neurotransmission modifying compound (e.g. AChEIs or the N-methyl-D-aspartate receptor antagonist) it may be expected based on the disclosure herein that the symptomatic homeostatic response may eventually be displaced by the response to the therapeutic compound (which may be either MT-containing, or otherwise).

In another aspect there is provided a method of therapeutic treatment of Alzheimer's disease or Mild Cognitive Impairment in a subject,
which subject has been selected as being non-responsive to treatment with a neurotransmission modifying compound which is a modifier of the activity of acetylcholine or glutamate neurotransmitters,
which method comprises administering to said subject a therapeutic methylthioninium (MT)-containing compound,
wherein the MT-containing compound is an LMTX compound of the following formula:

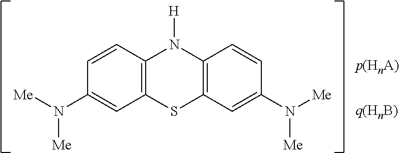

wherein each of HₙA and HₙB (where present) are protic acids which may be the same or different,
and wherein p=1 or 2; q=0 or 1; n=1 or 2; (p+q)×n=2,

or a hydrate or solvate thereof,
followed by treatment with said neurotransmission modifying compound.

The Alzheimer's disease may be mild Alzheimer's disease. The LTMX may be used for a treatment timeframe with co-administration with the neurotransmission modifying compound, followed by treatment with the neurotransmission modifying compound without the LMTX, wherein optionally the treatment timeframe with co-administration is at least 2 months, but can be at least 6, 12 or 18 months e.g. up to 24 months.

Thus there is provided use of an LMTX compound (or combination of LMTX compound and neurotransmission modifying compound) to enhance responsiveness to a neurotransmission modifying compound of a subject, particularly a subject who is otherwise been considered to be non-responsive to such a neurotransmission modifying compound.

Some aspects and embodiments of the invention will now be discussed in more detail.

Patient Selection

As explained above, according to context, the selection in relation to the present invention relates to subjects and their status regarding prior exposure to neurotransmission modifying compounds which are modifiers of the activity of acetylcholine or glutamate neurotransmitters. Such compounds are AChEIs or the N-methyl-D-aspartate receptor antagonist memantine. Examples of acetylcholinesterase inhibitors include Donepezil (Aricept™), Rivastigmine (Exelon™) or Galantamine (Reminyl™). An example of an NMDA receptor antagonist is Memantine (Ebixa™, Namenda™).

In some aspects the subject group may be entirely naïve to these other treatments, and have not historically received one or both of them.

In other aspects the subject group may have historically received one or both of these treatments, but ceased that medication at least 2, 3, 4, 5, 6, 7, 8, 12, or 16 weeks, or optionally at least 1, 2, 3, 4, 5 or 6 months etc. prior to treatment with an MT compound according to the present invention. By “ceased” is meant either stopped at a point in time, or is titrated steadily to zero.

In other aspects there may be an initial period of co-administration of the therapeutic and neurotransmission modifying compound, during which the dosage of neurotransmission modifying compound is “titrated down” to zero as explained above.

In other aspects, the subject group may be selected as being non-responsive to treatment with a neurotransmission modifying compound which is a modifier of the activity of acetylcholine or glutamate neurotransmitters i.e. having not showed any significant clinical benefit to one or both of these symptomatic treatments, Typically in such cases the drugs would be deemed unsuitable for the patient group, but as explained herein, prior or combination treatment with LMTX may create a utility for later use of these symptomatic treatments.

Aspects of the present invention include the active step of selecting the subject group according to these criteria. Such active selection will typically involve specific enquiry to the subject or care-giver, and appropriate action including (1) exclusion, if a subject is to continue treatment with the neurotransmission modifying compounds without ceasing prior to or during a first timeframe with the therapeutic, or (2) acceptance if they are naïve to that treatment, or are able to cease treatment for sufficient time, either prior to administration of the therapeutic, or during a period of co-administration when the dosage of neurotransmission modifying compound is “titrated down” to zero as explained above.

Combination Therapy

In the present invention there is at least one timeframe during which treatment with the therapeutic compounds (which may or may not be MT-containing) is conducted without treatment with the symptomatic treatment (neurotransmission modifying compound).

This treatment may be a monotherapy i.e. with no other active treatment, or it may be a combination therapy (with yet another active treatment e.g. a non-symptomatic one).

After the initial treatment the therapeutic compounds may be combined (or recombined) with the symptomatic treatment (neurotransmission modifying compound).

Thus the term “treatment” includes “combination” treatments, in which two or more treatments to treat the relevant disease are are combined, for example, sequentially or simultaneously.

In combination treatments, the agents (i.e., e.g. MT-containing, or not containing, compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

Thus in one embodiment administration of the MT-compound is commenced or continued in subjects who have not received (for a period of time) AChEIs or memantine, but then treatment with such AChEIs or memantine treatment is started or re-started after a period of treatment with the MT compound, for example after around 2 months or more preferably 6 months of treatment with the MT compound.

Typically such symptomatic co-treatment would be commenced (or re-commenced) when a physician judged that such would benefit the subject.

Disease Modifying and Symptomatic Treatments

In the methods of the present invention the therapeutic MT-containing or not containing compound is preferably “disease modifying” (or putatively “disease modifying”) as distinct from symptomatic in action.

This property may be inferred at the outset, for example, on the basis of a known or expected effect on the etiology of the disorder in question.

Symptomatic agents defer or mitigate the symptoms of the disease without affecting the fundamental disease process and do not change the rate of longer term decline after an initial period of treatment. If, after withdrawal, the patient reverts to where they would have been without treatment, the treatment is deemed to be symptomatic (Cummings, J. L. (2006) Challenges to demonstrating disease-modifying effects in Alzheimer's disease clinical trials. Alzheimer's and Dementia, 2:263-271).

AChEIs and the N-methyl-D-aspartate receptor antagonist memantine are examples of symptomatic treatments. Tau aggregation inhibitors are examples of disease modifying treatments.

Disease modification may also be inferred from clinical evidence, e.g. if after withdrawal from treatment the patient reverts to where they would have been without treatment, the treatment may be deemed to be symptomatic rather than disease-modifying.

Alternatively (in relation to trials) if a patient randomised late to active treatment is never able to catch up with a patient randomised early to active treatment, then the treatment is deemed to modify disease.

LMTX Compounds

Some aspects of the invention concern “LMTX” compounds, for example of the type described in WO2007/110627 or WO2012/107706.

Thus the compound may be selected from compounds of the following formula, or hydrates or solvates thereof:

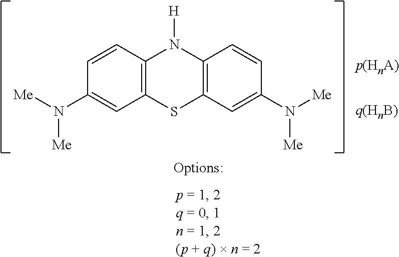

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

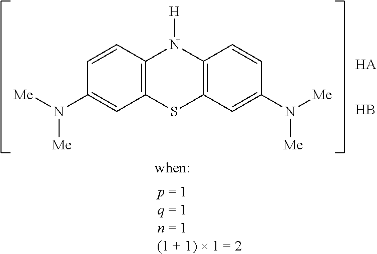

However preferably the salt is not a mixed salt, and has the following formula:

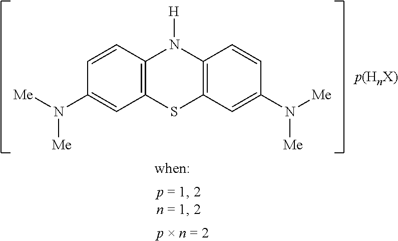

wherein each of HₙX is a protic acid, such as a di-protic acid or mono-protic acid.

In one embodiment the salt has the following formula, where H₂A is a di-protic acid:

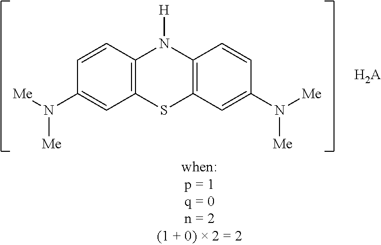

Preferably the salt has the following formula which is a bis monoprotic acid:

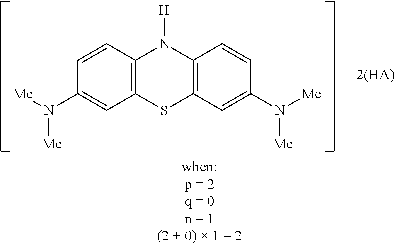

Examples of protic acids which may be present in the LMTX compounds used herein include:

Inorganic acids: hydrohalide acids (e.g., HCl, HBr), nitric acid (HNO₃), sulphuric acid (H₂SO₄)

Organic acids: carbonic acid (H₂CO₃), acetic acid (CH₃OOOH), methanesulfonic acid, 1,2-ethanedisulfonic acid, ethanesulfonic acid, naphthalenedisulfonic acid, p-toluenesulfonic acid,

Preferred acids are monoprotic acid, and the salt is a bis(monoprotic acid) salt.

A preferred MT compound is LMTM:

| | | | |
|---|---|---|---|
| 1 | 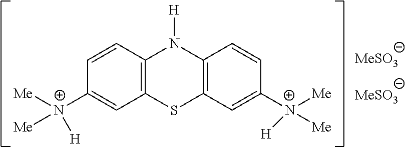 | LMT.2MsOH (LMTM) | 477.6 (1.67) |

The anhydrous salt has a molecular weight of around 477.6. Based on a molecular weight of 285.1 for the LMT core, the weight factor for using this MT compound in the invention is 1.67. By “weight factor” is meant the relative weight of the pure MT containing compound vs. the weight of MT which it contains.

Other weight factors can be calculated for example MT compounds herein, and the corresponding dosage ranges can be calculated therefrom.

Therefore the invention embraces a total daily dose of around 0.8 to 33 mg/day of LMTM.

More preferably around 6 to 12 mg/day of LMTM total dose is utilised, which corresponds to about 3.5 to 7 mg MT.

Other example LMTX compounds are as follows. Their molecular weight (anhydrous) and weight factor is also shown:

| | | | |
|---|---|---|---|
| 2 | 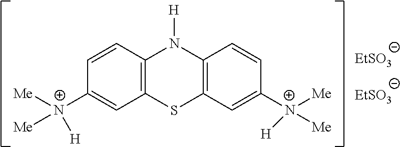 | LMT.2EsOH | 505.7 (1.77) |
| 3 | 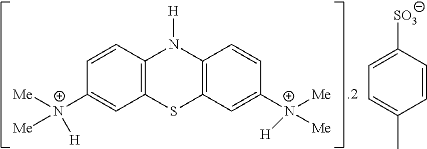 | LMT.2TsOH | 629.9 (2.20) |
| 4 | 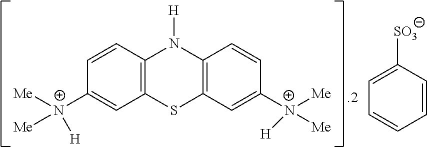 | LMT.2BSA | 601.8 (2.11) |
| 5 | 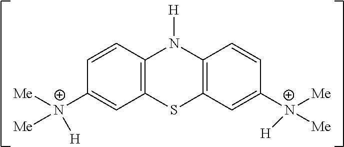 | LMT EDSA | 475.6 (1.66) |
| | 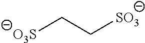 | | |
| 6 | 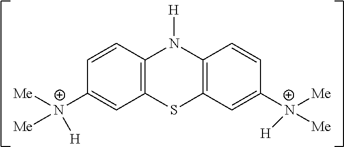 | LMT.PDSA | 489.6 (1.72) |
| | 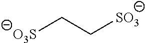 | | |
| 7 | 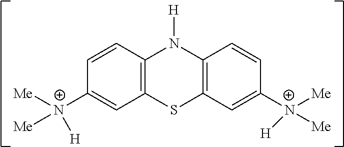 | LMT.NDSA | 573.7 (2.01) |
| | 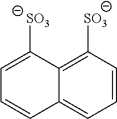 | | |
| 8 | 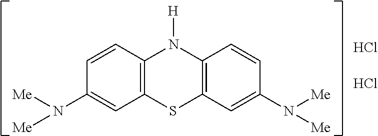 | LMT.2HCl | 358.33 (1.25) |

In the various aspects of the invention described herein (as they relate to an MT-containing compound) this may optionally be any of those compounds described above:

In one embodiment, it is compound 1.

In one embodiment, it is compound 2.

In one embodiment, it is compound 3.

In one embodiment, it is compound 4.

In one embodiment, it is compound 5.

In one embodiment, it is compound 6.

In one embodiment, it is compound 7.

In one embodiment, it is compound 8.

Or the compounds may be a hydrate, solvate, or mixed salt of any of these.

LMTX Dosages

Based on prior and concurrent results using LMTM in the treatment of disease, it can be concluded that MT dosages in the range 2-80 or 100 mg/day could be beneficial for the diseases described herein.

More specifically, further analysis of the concentration-response for LMTM in relation to the treatment of disease supports the proposition that a preferred dose is at least 2 mg/day, and doses in the range 20-40 mg/day, or 20-60 mg/day would be expected to maximise the cognitive benefit while nevertheless maintaining a desirable profile in relation to being well tolerated with minimal side-effects.

Thus in one embodiment the total MT dose may be from around any of 2, 2.5, 3, 3.5, 4 mg to around any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg.

An example dosage is 2 to 60 mg.

An example dosage is 20 to 40 mg.

Further example dosages are 8 or 16 or 24 mg/day.

The subject of the present invention may be an adult human, and the dosages described herein are premised on that basis (typical weight 50 to 70 kg). If desired, corresponding dosages may be utilised for subjects outside of this range by using a subject weight factor whereby the subject weight is divided by 60 kg to provide the multiplicative factor for that individual subject.

As will be appreciated by those skilled in the art, for a given daily dosage, more frequent dosing will lead to greater accumulation of a drug.

The present inventors have derived estimated accumulation factors for MT as follows:

| | Observed plasma | Relative |
|---|---|---|
| Dosing | accumulation for MT | accumulation |
| Once daily | 1.29^{extrapolated} | 1 |
| Twice daily | 1.47 | 1.13 |
| Three-times daily | 1.65 | 1.28 |

For example, considering a total daily dose of 3.5 to 7 mg MT:

When given as a single daily dose, this may equate to an accumulation of MT in plasma of 4.5 to 8

When split b.i.d., this may equate to an accumulation of MT in plasma of 5.1 to 10.3

When split t.i.d., this may equate to an accumulation of MT in plasma of 5.8 to 11.6

Therefore in certain embodiments of the invention, the total daily dosed amount of MT compound may be lower, when dosing more frequently (e.g. twice a day [b.i.d.] or three times a day [t.i.d.]).

In one embodiment, LMTM is administered around 9 mg/once per day; 4 mg b.i.d.; 2.3 mg t.i.d (based on weight of LMTM).

In one embodiment, LMTM is administered around 34 mg/once per day; 15 mg b.i.d.; 8.7 mg t.i.d (based on weight of LMTM).

The MT compound of the invention, or composition comprising it, is administered to a subject orally.

In some embodiments, the MT compound is administered as a composition comprising the LMTX compound as described herein, and a pharmaceutically acceptable carrier, diluent, or excipient.

The term “pharmaceutically acceptable,” as used herein, pertains to compounds, ingredients, materials, compositions, dosage forms, etc., which are suitable for use in contact with the tissues of the subject in question without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Compositions comprising LMTX salts are described in several publications e.g. WO2007/110627, WO2009/044127, WO2012/107706, WO2018019823 and WO2018041739.

In some embodiments, the composition is a composition comprising at least one LMTX compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

In some embodiments, the composition is a tablet.

In some embodiments, the composition is a capsule.

In some embodiments, said capsules are gelatine capsules.

In some embodiments, said capsules are HPMC (hydroxypropylmethylcellulose) capsules.

In some embodiments, the amount of MT in the unit 2 to 60 mg.

In some embodiments, the amount of MT in the unit 10 to 40, or 10 to 60 mg.

In some embodiments, the amount of MT in the unit 20 to 40, or 20 to 60 mg.

An example dosage unit may contain 2 to 10 mg of MT.

A further example dosage unit may contain 2 to 9 mg of MT.

A further example dosage unit may contain 3 to 8 mg of MT.

A further preferred dosage unit may contain 3.5 to 7 mg of MT.

A further preferred dosage unit may contain 4 to 6 mg of MT.

In some embodiments, the amount is about 2, 2.5, 3, 3.5, 4, 5, 6, 7, 8, 9, 10, 11, 12, 13, 14, 15, 16, 17, 18, 19 or 20 mg of MT.

Using the weight factors described or explained herein, one skilled in the art can select appropriate amounts of an MT containing compound to use in oral formulations.

As explained above, the MT weight factor for LMTM is 1.67. Since it is convenient to use unitary or simple fractional amounts of active ingredients, non-limiting example LMTM dosage units may include about 3, 3.5, 4, 5, 6, 7, 8, 9, 10, 15, 16, 17, 34, 50, 63 mg etc.

Non MT-Containing Compounds

Some aspects of the invention concern therapeutic compounds used or intended for the treatment of Alzheimer's disease or Mild Cognitive Impairment which do not contain methylthioninium (MT).

Such compounds may be intended to be disease modifying or symptomatic. The primary pathologies in AD that have been targeted for disease modification are tau and amyloid. Such drugs include those intended to reduce amyloid-β (Aβ) production, promote Aβ clearance, as well as drugs to increase tau modification and inhibit aggregation.

None limiting examples of compounds which are reported to be suitable for such treatments are as follows:

Drugs which Target Processing of Amyloid-β (Aβ)

Drugs affecting Aβ include BACE inhibitors, immunotherapeutic approaches (both passive and active immunotherapies), α-secretase inhibitors or modulators, γ-secretase activators and Aβ aggregation inhibitors, including metal chelators (such as PBT2) (Panza et al., 2019).

Drugs which Target Tau Protein

Drugs that target tau protein include inhibitors of tau aggregation (e.g. daunorubicin, Congo red, anthraquinones, benzothiazoles, cyanin dyes, phenylthiazolyl-hydrazides, N-phenylamines, rhodanine, polyphenols, porphyrins, quinoxalines, aminthienopyridazines, oleocanthal, curcumin, and others); microtubule stabilisers (e.g. epithilone D, dictyostatin; TPI-287); kinase inhibitors to prevent the phosphorylation of tau (e.g. Tideglusib, sodium selenate, LiCl); acetylation inhibitor (salsalate); deglycosylation inhibitor (MK8719); and phosphodiesterase 4 inhibitor (BPN14770) (Congdon and Sigurdsson, 2015; see especially FIG. 4). In addition tau immunotherapy approaches are in clinical trials (e.g. AADvac1, ACI-35, RG7345).

Drugs that target neural regeneration-based strategies include, for example, ciliary neurotrophic factor-based peptides (Kazim and Iqbal, 2017).

Drugs that have multiple putative AD-related targets, such as Dimebon (or Latrepirdine) (Ustyugov et al., 2018).

Another proposed therapeutic approach for AD is via miRNA inhibitors (Jaber et al., 2019) to target synaptic communication, defects in assembly and enhanced clearance of either Aβ or tau, deficits in neurotrophic support, proliferation of neuroinflammatory signalling and alterations in the innate immune response. miRNA inhibitors are single-stranded, modified RNAs which specifically inhibit endogenous miRNA function in cells. Neural miRNAs are involved at various stages of synaptic development, including dendritogenesis (involving miR-132, miR-134 and miR-124), synapse formation and synapse maturation (where miR-134 and miR-138 are thought to be involved).

Drugs that have Symptomatic Effects on AD

Although less preferred, the present invention may also be applied, in principle, to therapies even which are not disease-modifying. For example different symptomatic treatments which may otherwise be affected by the homeostatic response described herein e.g. psychostimulants such as amphetamines (see e.g. Dolder, Christian R., Lauren Nicole Davis, and Jonathan McKinsey. “Use of psychostimulants in patients with dementia.” Annals of Pharmacotherapy 44.10 (2010): 1624-1632).

MCI

Mild Cognitive Impairment is recognised as a valid disease target by the FDA. It is defined by having a minor degree of cognitive impairment not yet meeting clinical criteria for a diagnosis of dementia.

Representative criteria for syndromal MCI include features listed below:

A. The patient is neither normal nor demented.

B. There is evidence of cognitive deterioration shown by either objectively measured decline over time and/or subjective report of decline by self and/or informant in conjunction with objective cognitive tests (e.g. secondary tests if memory).

C. Activities of daily living are preserved and complex instrumental functions are either intact or minimally impaired.

(See also Winblad, B. et al. (2004) “Mild cognitive impairment—beyond controversies, towards a consensus: report of the International Working Group on Mild Cognitive Impairment.” J. Intern. Med. 256: 240-246).

As used above, the term “dementia” refers to a psychiatric condition in its broadest sense, as defined in American Psychiatric Association: Diagnostic and Statistical Manual of Mental Disorders, Fourth Edition, Washington, D.C., 1994 (“DSM-IV”). The DSM-IV defines “dementia” as characterized by multiple cognitive deficits that include impairments in memory and lists various dementias according to presumed etiology. The DSM-IV sets forth a generally accepted standard for such diagnosing, categorizing and treating of dementia and associated psychiatric disorders.

MCI subjects for whom the present invention may preferably be used may be those with less than or equal to MMSE 24, 25, 26, 27, 28 or 29, more preferably less than or equal to MMSE 24, 25, 26, most preferably less than or equal to MMSE 24 or 25. The MMSE test is discussed in more detail hereinafter.

Another aspect of the present invention pertains to the therapeutic compounds as described herein for use the methods as described above e.g. methods of therapeutic treatment of Alzheimer's disease or Mild Cognitive Impairment in a subject, or methods for assessing the efficacy of the compounds where they are putatively therapeutic for Alzheimer's disease or Mild Cognitive Impairment in a subject.

Another aspect of the present invention pertains to use of a therapeutic compounds described herein in the manufacture of a medicament for use in such methods.

Certain aspects of the invention concern methods for assessing the efficacy of a compound which does not contain methylthioninium (MT) which is putatively therapeutic for AD or MCI in a subject.

WO2009/060191 concerns the design of clinical trials for putative therapeutics for neurodegenerative disorders and is specifically incorporated herein by cross reference in relation to the general conduct and means of analysis of such trials.

The methods of the invention are generally concerned with clinical trials for testing a pharmaceutical (or putative pharmaceutical e.g. an investigational medicinal product (IMP)), although they may also be employed for managing therapy whereby new treatment regimens employing the pharmaceutical are being tested or compared for their efficacy.

Thus the methods herein may be used for performing a clinical trial, or for providing a system for performing said trial.

Such a system may be for assessing the efficacy of a compound which does not contain methylthioninium (MT) which is putatively therapeutic for Alzheimer's disease or Mild Cognitive Impairment in a subject, the system comprising the steps of:

(a) selecting a subject group which consists only of:
(i) subjects who have not historically received treatment with a neurotransmission modifying compound which is a modifier of the activity of acetylcholine or glutamate neurotransmitters; and
(ii) subjects who have historically received treatment with a neurotransmission modifying compound which is a modifier of the activity of acetylcholine or glutamate neurotransmitters, but ceased that treatment at least 3, 4, 5, 6, 7, or 8 weeks prior to said assessment;

(b) stratifying the subject groups into at least 2 sub-groups according to a baseline indicator of likely disease progression,

(c) selecting a treatment timeframe over which members of each subject group are to be treated with the non-MT-containing compound,

(d) selecting psychometric and optionally physiological outcome measures to be derived for each treated patient group and a comparator arm of said sub-groups which is optionally a placebo or minimal efficacy comparator arm,
whereby the efficacy measure for the pharmaceutical may be derived from a comparison of the treated patient groups and the comparator arm.

The methods are particularly suitable to providing evidence of clinical efficacy suitable for meeting appropriate regulatory standards for marketing e.g. as required by the US Food and Drug Administration (FDA) or European Agency for the Evaluation of Medicinal Products (EMEA).

Subject Groups and Predictive Indicators of Likely Disease Progression

The subject group will typically be patients diagnosed with the disorder in question using conventional criteria (e.g. National Institute of Neurological and Communicative Disorders and Stroke-Alzheimer's Disease and Related Disorders Association [NINCDS-ADRDA]; DSM-IV etc.

The DSM-IV sets forth a generally accepted standard for such diagnosing, categorizing and treating of dementia and associated psychiatric disorders.

In the methods of the invention the subject group is itself stratified according to baseline indicators of likely disease progression. This in turn can be assessed in terms of disease severity.

Preferably, in AD, disease severity is assessed using the so-called Clinical Dementia Rating (CDR) scale (Hughes, C. P., Berg, L., Danziger, W. L., Coben, L. A., Martin, R. L. (1982) A new clinical scale for the staging of dementia. British Journal of Psychiatry, 140:566-572; Morris, J. C. (1993) The Clinical Dementia Rating (CDR): Current version and scoring rules. Neurology, 43:2412-2414).

The CDR may optionally be informed by a structured clinical examination e.g. the short version of the CAMDEX (Roth, M., Tym, E., Mountjoy, C. Q., Huppert, F. A., Hendrie, H., Verma, S. & Goddard, R. (1986) CAMDEX. A standardised instrument for the diagnosis of mental disorder in the elderly with special reference to the early detection of dementia. British Journal of Psychiatry, 149:698-709). Alternatively the CDR may be informed by the structured psychiatric interview as defined by Hughes et al. (1982) or Morris (1993).

For example, sub-groups may be formed from subjects having a CDR rating of 1 (mild sub-group) or 2 (moderate sub-group).

Disease severity may also be assessed e.g. using the “Braak staging” methods described in WO02/075318. Sub-groups may then be formed from subjects having Braak stage up to 1, 2, 3 and 4, and so on.

As noted above, the subject groups are additionally actively selected in relation to their prior exposure to certain symptomatic treatments.

The sub-groups will generally be tested in parallel.

Treatment Time Frame

Treatment timeframes in relation to AD or MCI may be selected based on the disease severity of the subgroup. Typical time frames for a clinical trial according to the present invention may be more than or equal to 12 weeks, 16 weeks, 24 weeks, 25 weeks, 36 weeks, 50 weeks, 100 weeks (or more than or equal to 3 months, 4 months, 6 months, 9 months, 12 months, 24 months and so on). Depending on the time frame thus selected, the present invention provides for the use of novel measures or analysis to derive more accurate measures of pharmaceutical efficacy.

Preferred trials may be less than the periods above, e.g. less than 9, 6, 5, 4, or 3 months.

For example for shorter time scales, and e.g. in patients having relatively low disease severity at baseline (where cognitive decline as measured by psychometric outcome measures may be masked by cognitive reserve) it may be preferable to use additional physiological outcome measures and\or more sensitive psychometric outcome measures.

Other trials may be more than 6 or 12 months.

For example for longer time scales (and e.g. in patients having relatively high disease severity at baseline, where “fit survivor” artifacts are more likely to occur), it may be preferable to use a linear imputation method for each individual discontinuing treatment to correct the analysis for the effect of discontinuation.

The time-frames may be same or different for the sub-groups.

Psychometric Outcome Measures

Psychometric outcome measures for use in the methods may be conventional ones, as accepted by appropriate regulatory bodies.

For AD, the Alzheimer's Disease Assessment Scale-cognitive subscale [ADAS-cog] is preferred (Rosen W G, Mohs R C, Davis K L. A new rating scale for Alzheimer's disease. Am J Psychiatry. 1984 November; 141(11):1356-64).

Another standardised test is the Mini-Mental State Examination [MMSE] which was proposed as a simple and quickly administered method for grading cognitive function (Folstein M F, Folstein S E & McHugh P R. ‘Mini-mental state’. A practical method for grading the cognitive state of patients for the clinician. Journal of Psychiatric Research 1975 12 189-198). The MMSE is the most widely used cognitive screening instrument for the detection of cognitive dysfunction due to dementia in geriatric and psychiatric patients (Tombaugh T N & McIntyre N J. The mini-mental state examination: a comprehensive review. Journal of the American Geriatric Society 1992 40 922-935). The MMSE evaluates orientation, memory, attention and language functions.

The assessment or analysis of psychometric outcome measures may include the step of performing a linear imputation analysis on the available psychometric scores of individual subjects discontinuing treatment. This may involve a straight line per-subject extrapolation fitted to the graph of said scores (e.g. ADAS-cog change scores).

Physiological Outcome Measures

As described herein, in addition to psychometric testing, the present inventors provide for the use of neurophysiological outcome measures, e.g. by way of analysis of changes in functional brain scans. This increases the sensitivity of analysis of disease modifying treatment when testing even for relatively short time periods, e.g. 3 or 4 months.

Scans may employ SPECT (Single Photon Emission Tomography) with the ligand ^{99m}Tc-HMPAO, or reductions in cerebral glucose uptake as measured by PET (Positron Emission Tomography) using ¹⁸fluoro-deoxyglucose (FDG), in the temporo-parietal association neocortex in AD.

The use of such scans generally is well known in the art for diagnosis (see e.g. Talbot, P. R., Lloyd, J. J., Snowden, J. S., Neary, D., Testa, H. J. (1998) A clinical role for 99mTc-HMPAO SPECT in the investigation of dementia? Journal of Neurology, Neurosurgery and Psychiatry, 64:306-313; Masdeu, J. C., Zubieta, J. L., Arbizu, J. (2005) Neuroimaging as a marker of the onset and progression of Alzheimer's disease. Journal of the Neurological Sciences, 236:55-64) and in examining response to therapy (Venneri, A., Shanks, M. F., Staff, R. T., Pestell, S. J., Forbes, K. E., Gemmell, H. G., Murray, A. D. (2002) Cerebral blood flow and cognitive responses to rivastigmine treatment in Alzheimer's disease. NeuroReport; 13:83-87).

Structural imaging based on magnetic resonance is an integral part of the clinical assessment of patients with suspected Alzheimer dementia. Atrophy of medial temporal structures is considered to be a valid diagnostic marker at the mild cognitive impairment stage. In addition, rates of whole-brain and hippocampal atrophy are sensitive markers of neurodegeneration (Frisoni et al., 2010, Nature Reviews Neurology 6:67-77).

Conversely, ventricular enlargement detected using magnetic resonance imaging, can also be used a measure of Alzheimer's disease progression (Nestor et al., 2008, Brain 131: 2443-2454).

It will be appreciated that, in addition to these techniques, any other applicable methodology which permits direct measure of pathological burden in the brain may be employed.

Comparator Arm

Following randomisation of the sub-groups, some subjects in each sub-group will be selected for comparator treatment. Preferably this will be a placebo (non-treatment) or minimal efficacy comparator arm of the trial. However alternative designs, where it is unethical to withhold already existing treatments, involve randomisation to alternative active treatment arms either singly or in some prespecified combination.

Final Efficacy Measure

Typically this will ultimately be based on a psychometric outcome, based on appropriate statistical methods for comparing the relevant treatment and placebo arms. This may optionally be ANCOVA or if necessary to achieve more power a linear-mixed effects approach (Petkova, E. and Teresi, J. (2002) Some statistical issues in the analysis of data from longitudinal studies of elderly chronic care populations. Psychosomatic Medicine, 64:531-547) such as shown in Examples below. Physiological outcome measures may also be employed in the final analysis. Suitable clinical end points demonstrating efficacy in this respect can be selected by those skilled in the art, in the light of the present disclosure.

Specifically, efficacy can be demonstrated where there is a statistically significant difference between subjects randomised to active treatment at some specified dose and subjects receiving the comparator treatment, dose or placebo.

The therapeutic compounds described herein (e.g. defined dose of MT-containing compound plus optionally other ingredients) may be provided in a labelled packet along with instructions for their therapeutic or prophylactic use, for example their use in monotherapy or combination therapy as discussed above.

In one embodiment, the pack is a bottle, such as are well known in the pharmaceutical art. A typical bottle may be made from pharmacopoeial grade HDPE (High-Density Polyethylene) with a childproof, HDPE push-lock closure and contain silica gel desiccant, which is present in sachets or canisters. The bottle itself may comprise a label, and be packaged in a cardboard container with instructions for use (as per the methods describe and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding the maximum permitted daily dosage of the compositions as described herein—for example based on once daily, b.i.d., or t.i.d.

Said label or instructions may provide information regarding the suggested duration of treatment.

Salts and Solvates

Although the LMTX containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

Treatment and Prophylaxis

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition.

The invention also embraces treatment as a prophylactic measure.

The term “therapeutically-effective amount” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The therapeutic compounds will typically be administered in a “therapeutically-effective amount” or “prophylactically effective amount”.

The term “prophylactically effective amount” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. Pharmacokinetic-pharmacodynamic response on the ADAS-cog scale over 65 weeks in patients with mild to moderate AD taking LMTM at a dose of 8 mg/day and categorized by co-medication status with AD-labelled treatments.

FIG. 2. Treatment effects of LMTM alone or following chronic pretreatment with rivastigmine in wild-type mice on hippocampal levels of acetylcholine (A) or synaptophysin levels measured immunohistochemically as the mean value for hippocampus, visual cortex, diagonal band and septum (B). (**, p<0.01; ***, p<0.001).

FIG. 3. Treatment effects of LMTM alone or following chronic pretreatment with rivastigmine in tau transgenic L1 mice on levels of (A) SNARE complex proteins (SNAP25, syntaxin and VAMP2) and (B) α-synuclein measured immunohistochemically as the mean value for hippocampus, visual cortex, diagonal band and septum. (*, p<0.05; ***, p<0.001; ****, p<0.0001).

FIG. 4. Treatment effects of LMTM alone or following chronic pretreatment with rivastigmine in tau transgenic L1 mice on complex IV activity measured relative to citrate synthetase activity in brain mitochondria. (*, p<0.05).

FIG. 5. Treatment effects of LMTM alone or following chronic pretreatment with rivastigmine in tau transgenic L1 mice compared with vehicle-treated wild-type mice on levels of tau immunoreactivity (relative optical density) (A) and neurons immunoreactive for choline acetyltransferase (B) in vertical diagonal band. (*, p<0.05; **, p<0.01; ***, p<0.001).

FIG. 6. Summary schema of treatment effects of LMT which are subject to dynamic modulation by chronic pretreatment with the acetylcholinesterase inhibitor (AChEI) rivastigmine (with particular focus on changes in mitochondrial metabolism and presynaptic proteins) and tau aggregation inhibitor activity. Combined treatment with AChEI does not impair LMT effects on tau aggregation pathology. By contrast, the combination prevents the increases in synaptic proteins, ACh release and increased complex IV activity that are seen following treatment with LMTM alone.

FIG. 7. Effect of combined administration of Memantine and LMTM on problem solving deficits in female Line 1 mice, aged 5.5 months at the beginning of the study. Memantine was administered at 20 mg/kg and LMTM at 15 mg/kg. Mice were treated with vehicle or Memantine for 5 weeks prior to Memantine plus LMTM treatment for 6 weeks, with the mice being tested in the water maze task over weeks 10 and 11.

FIG. 8. Withdrawal following 18 month LMTM 8 mg/day monotherapy (ADAS-cog). As can be seen in the subjects in the high exposure (Cmax) group continue to decline at same rate following LMTM withdrawal (change=0.73; p-value=0.1651). This supports a persistent disease-modifying change in rate of cognitive decline following the LMTX treatment period.

The subjects exposed to a lower exposure suffered a large decline (change=2.29; p-value=0.0011) implying that the LMTX benefit in that group may be at least partly symptomatic, and hence not persistent.

FIG. 9. Withdrawal following 18 month LMTM 8 mg/day as an “add on” to existing symptomatic therapy (AChEI, Memantine) (ADAS-cog). As can be seen in the subjects in the high exposure (Cmax) group show improvement following LMTM withdrawal (change=−0.98 p-value=0.0021). This is consistent with either a disease-modifying effect during LMTM treatment which results in an improved response to symptomatic treatment alone, or a negative effect on the symptomatic treatment exhibited by LMTM during the LMTX treatment period.

The subjects exposed to a lower exposure continue to decline at same rate following withdrawal (change=0.51, p-value=0.0855).

### EXAMPLES

### Example 1—Provision of MT-Containing Compounds

Methods for the chemical synthesis of the MT-containing compounds described herein are known in the art. For example:

Synthesis of compounds 1 to 7 can be performed according to the methods described in WO2012/107706, or methods analogous to those.

Synthesis of compound 8 can be performed according to the methods described in WO2007/110627, or a method analogous to those.

### Example 2—Investigation of Interference Between LMTM and Symptomatic Treatments in a Tau Transgenic Mouse Model

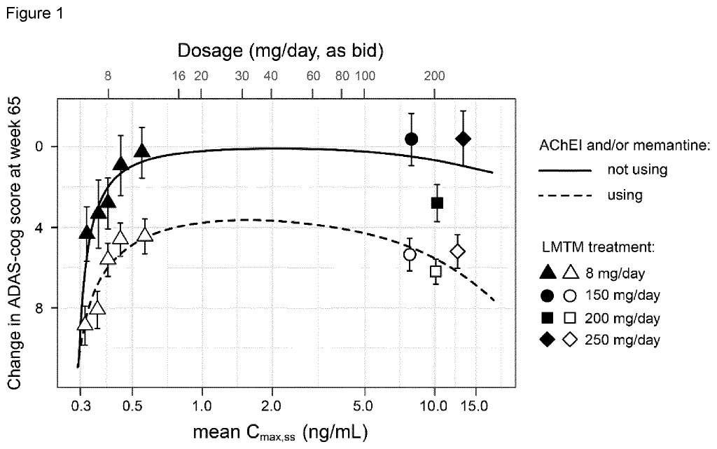

FIG. 1 illustrates the interference which was established then LMTM was taken in combination with symptomatic treatments.

We have undertaken work in a well-characterised tau transgenic mouse model (Line 1, “L1”; (Melis et al., 2015b)) with the aim of understanding the mechanisms responsible for the reduced efficacy of LMTM as an add-on to symptomatic treatments.

In summary, our findings suggest that homeostatic mechanisms downregulate multiple neuronal systems at different levels of brain function to compensate for the chronic pharmacological activation induced by symptomatic treatments.

Compared with LMTM given alone, the effect of this downregulation is to reduce neurotransmitter release, levels of synaptic proteins, mitochondrial function and behavioural benefits if LMTM is given against a background of chronic prior exposure to acetylcholinesterase inhibitor. Therefore, the interference in treatment efficacy, first seen clinically, has a clear neuropharmacological basis that can be reproduced in a tau transgenic mouse model.

Importantly, the homeostatic effects we have identified are likely to have more general relevance for the conduct of disease-modifying trials, or indeed other kinds of therapeutic compound-trials, in AD or MCI, that need not be restricted to tau aggregation inhibitors.

In the L1 mouse model which was used in some of the present studies, there is over-expression of a three-repeat tau fragment encompassing residues 296-390 of the 2N4R tau isoform under the control of the Thy 1 promotor in an NMRI mouse strain (WO2002/059150). This fragment corresponds to the segment of tau first identified within the proteolytically stable core of the PHF (Wischik et al., 1988a; Wischik et al., 1988b) and encompasses the fragment 306-378 recently confirmed by cryo-electronmicroscopy of PHFs in AD and tau filaments in Pick's disease (Fitzpatrick et al., 2017; Falcon et al., 2018).

Further features of the L1 mouse model include a prominent loss of neuronal immunoreactivity for choline acetyltransferase in the basal forebrain region, and a corresponding reduction in acetylcholinesterase in neocortex and hippocampus, indicative of reduction in acetylcholine. There is also an approximate 50% reduction in glutamate release for brain synaptosomal preparations from L1 mice compared with those from wild-type mice. In these respects, therefore, L1 mice also model the neurochemical impairments in cholinergic (Mesulam, 2013; Pepeu and Grazia Giovannini, 2017) and glutamatergic (Revett et al., 2013) function that are characteristic of AD.

Underlying these impairments in neurotransmitter function, the L1 mouse model shows a disturbance in integration of synaptic proteins. Quantitative immunohistochemistry for multiple synaptic proteins in the basal forebrain (vertical diagonal band) shows that there is normally a high degree of correlation in levels of proteins comprising the SNARE complex (e.g. SNAP-25, syntaxin, VAMP2; reviewed in Li and Kavalali, 2017), and the vesicular glycoprotein synaptophysin and α-synuclein in wild-type mice. These correlations are largely lost in L1 mice (Table 1). The only correlations that remain are between synaptophysin, syntaxin and VAMP2. Therefore, synaptic vesicular protein levels are no longer linked quantitatively to the proteins of the SNARE complex or α-synuclein. This suggests that the tau oligomer pathology of the L1 mice interferes with the functional integration between vesicular and membrane-docking proteins in the synapse.

TABLE 1
Correlations between levels of a range of presynaptic proteins in basal
forebrain (vertical diagonal band) measured immunochemically in (A)
wild-type mice or (B) tau transgenic L1 mice.

| | | | | |
|---|---|---|---|---|
| α-Synuclein | SNAP25 | Syntaxin | VAMP2 | Synaptophysin |

A Wild-type mice

| | | | | | |
|---|---|---|---|---|---|
| α-Synuclein | | | | | |
| SNAP25 | * | | | | |
| Syntaxin | — | ** | | | |
| VAMP2 | — | * | * | | |
| Synaptophysin | — | ** | * | — | |
| Synapsin | — | — | — | — | — |

B L1 mice

| | | | | | |
|---|---|---|---|---|---|
| α-Synuclein | | | | | |
| SNAP25 | — | | | | |
| Syntaxin | — | — | | | |
| VAMP2 | — | — | — | | |
| Synaptophysin | — | — | — | * | |
| Synapsin | — | — | * | — | — |

Significance of correlations, by linear regression analysis, are denoted as * p < 0.05; ** p < 0.01; — no significance at p = 0.05.

### Example 3—Experimental Paradigms, Results and Discussion

Experimental Paradigms

The treatment schedule used to study the negative interaction between symptomatic treatments and LMTM was designed to model the clinical situation in which subjects are first treated chronically with a cholinesterase inhibitor or memantine before receiving LMTM. In what follows, we summarise some of the key results obtained for the AChEI, rivastigmine.

Wild-type and L1 mice (n=7-16 for each group) were pre-treated with rivastigmine (0.1 or 0.5 mg/kg/day) or memantine (2 or 20 mg/kg/day) or vehicle for 5 weeks by gavage. For the following 6 weeks, LMTM (5 and 15 mg/kg) or vehicle were added to this daily treatment regime, also by gavage. Animals were tested behaviourly during weeks 10 and 11 using a problem solving task in the open field water maze and then sacrificed for immunohistochemical and other tissue analyses.

Translating doses from mice to humans requires consideration of a number of factors. Although 5 mg/kg/day in mice corresponds approximately to 8 mg/day in humans in terms of Cₘₐₓ levels of parent MT in plasma, this dose is at the threshold for effects on pathology and behaviour. The higher dose of 15 mg/kg/day is generally required for LMTM to be fully effective in the L1 mouse model (Melis et al., 2015a). This may relate to the much shorter half-life of MT in mice (4 hours) compared to humans (37 hours in elderly humans). Tissue sectioned for immunohistochemistry was labelled with antibody and processed using Image J to determine protein expression densitometrically. Data are presented as Z-score transformations without units.

For measurement of acetylcholine (ACh) levels in hippocampus, animals (wild-type or L1) were treated with LMTM (5 mg/kg/day for 2 weeks) after prior treatment for 2 weeks with or without rivastigmine (0.5 mg/kg/day). Rivastigmine was administered subcutaneously with an Alzet minipump whereas LMTM was administered by oral gavage. Levels of ACh were measured in hippocampus using an implanted microdialysis probe and HPLC analysis of the extracellular fluid.

Data are presented as group averages and standard errors of mean and were analysed using parametric statistics, with alpha set to 0.05.

Experiments on animals were carried out in accordance with the European Communities Council Directive (63/2010/EU) with local ethical approval, a project license under the UK Scientific Procedures Act (1986), and in accordance with the German Law for Animal Protection (Tierschutzgesetz) and the Polish Law on the Protection of Animals.

Results

Effects of Treatment with LMTM and Rivastigmine in Wild-Type Mice

The effects of treatment with LMTM alone or on a chronic rivastigmine background are summarised in Table 2.

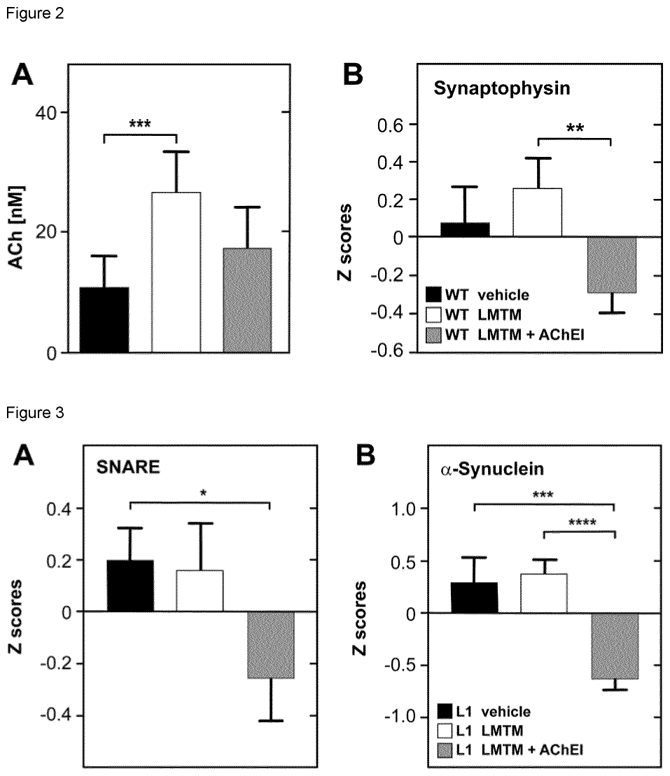

In wild-type mice, there was a significant, 2-fold increase in basal ACh levels in hippocampus following LMTM treatment, and a 30% reduction when mice received LMTM after prior treatment with rivastigmine (FIG. 2A).

There was also a 3-fold increase in synaptophysin levels measured in hippocampus, visual cortex, diagonal band and septum following LMTM treatment alone and a statistically significant reduction of the same magnitude when LMTM was given against a background of prior treatment with rivastigmine (FIG. 2B).

TABLE 2
Summary of treatment effects of LMTM given alone (5 or 15 mg/kg/day) or
following chronic pretreatment with rivastigmine (0.1 or 0.5 mg/kg/day) in
wild-type mice, given as approximate rounded percentages to indicate scale
and direction of change.

| | | Rivastigmine + |
|---|---|---|
| Effects in wild-type mice | LMTM alone | LMTM |
| ACh release | ↑ x 200% | ↓ x 30% |
| SNARE complex | — | — |
| Synaptophysin | ↑ x 300% | ↓ x 300% |
| α-Synuclein | — | — |
| Mitochondrial complex | — | — |
| IV | | |
| Behaviour | — | — |

Numbers in black signify treatment effects which reached statistical significance, ‘—’ indicates no effect.

Effects of Treatment with LMTM and Rivastigmine in Tau Transgenic L1 Mice

The activating effects of LMTM alone and the inhibitory effects of the combination with rivastigmine are larger and more generalised in the tau transgenic L1 mice than in the wild-type mice (see Table 3). LMTM alone produces significant increases in ACh release in the hippocampus, in glutamate release from brain synaptosomal preparations, in synaptophysin levels, in mitochondrial complex IV activity and in behavioural changes. None of these effects were seen when LMTM was preceded by chronic rivastigmine. Indeed, in the case of SNARE complex proteins (FIG. 3A) and synuclein (FIG. 3B), the reduction produced by the combination was to levels below those seen in the absence of LMTM treatment.

TABLE 3
Summary of treatment effects of LMTM given alone (5 or 15 mg/kg/day)
or following chronic pretreatment with rivastigmine (0.1 or 0.5 mg/
kg/day) in L1 mice, given as approximate rounded percentages to
indicate scale and direction of change.

| | | Rivastigmine + |
|---|---|---|
| Effects in L1 mice | LMTM alone | LMTM |
| ACh release | ↑ x 200% | ↓ x 30% |
| Glutamate release | ↑ x 200% | n/a |
| SNARE complex | — | ↓ x 300% |
| Synaptophysin | ↑ x 400% | ↓ x 300% |
| α-Synuclein | — | ↓ x 200% |
| Mitochondrial complex | ↑ x 50% | ↓ x 30% |
| IV | | |
| Behaviour | ↑ x 30% | ↓ x 20% |

Numbers in black signify treatment effects that reached statistical significance, those in grey were directional and n/a signifies that results are not yet available.

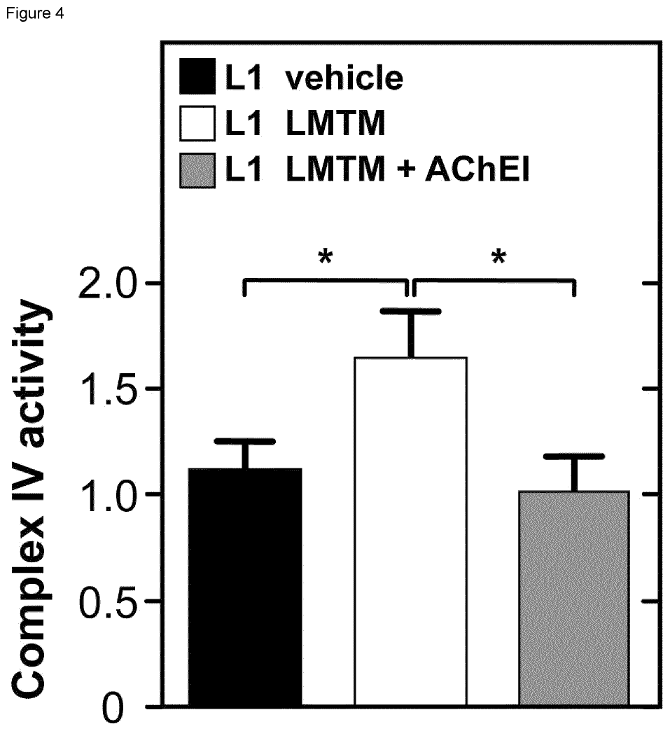

LMTM given alone produced significant enhancement of complex IV activity in brain mitochondria from tau transgenic L1 mice. Chronic pretreatment with rivastigmine also eliminated this effect (FIG. 4).

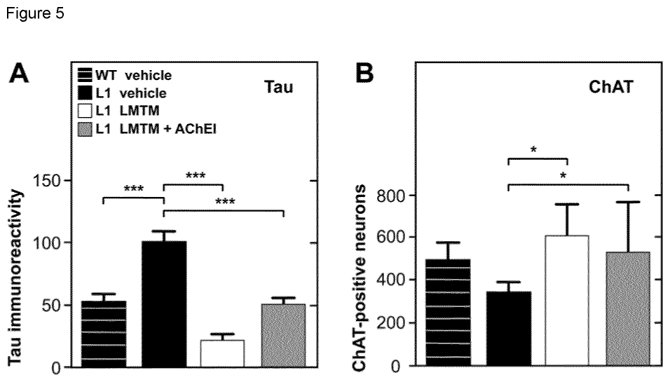

In contrast to the effects on neurotransmitter release, synaptic protein levels and mitochondrial complex IV activity, chronic pretreatment with rivastigmine has no effect on the primary action of LMTM as a tau aggregation inhibitor. As expected, immunoreactivity against the core tau unit of the PHF measured by optical density is elevated in tau transgenic L1 mice, and this was reduced following treatment with LMTM (FIG. 5A). Conversely, counts of ChAT-positive neurons are reduced in L1 mice and restored by treatment with LMTM (FIG. 5B). Both effects persist in L1 mice if LMTM is given after prior chronic treatment with rivastigmine.

Discussion of Example 3

The results presented here demonstrate that the reduction in efficacy of LMTM when given as an add-on to a symptomatic treatment in humans can be reproduced both in wild-type mice and in a tau transgenic mouse model. Therefore, it is based on neuropharmacological mechanisms that have the effect of altering how the brain responds to a disease-modifying treatment such as LMTM. The results imply that the differences in clinical response to LMTM as monotherapy or add-on therapy are likely to be explained by differences in the underlying neuropharmacology of LMTM in these two contexts (Gauthier et al., 2016; Wilcock et al., 2018). Alternative explanations based on the presumption that patients who are prescribed symptomatic treatments are somehow different from untreated patients fail for a number of reasons. The minor and variable differences in baseline severity between these two patient groups have been shown not to account for differences in treatment response (Gauthier et al., 2016; Wilcock et al., 2018). Apparent differences in rate of decline in treated and untreated MCI patients in the ADNI program (Schneider et al., 2011) disappear when severity at baseline is accounted for in the analysis (Wilcock et al., 2018). The presumption that untreated patients do not really have AD, or have a different form of AD, is also inconsistent with baseline neuroimaging data from subjects participating in the Phase 3 trials (Wilcock et al., 2018). Finally, as summarised for cognitive decline data in FIG. 1, we have shown recently that there are similar concentration-response relationships in monotherapy and add-on therapy subjects, but that the treatment effects are consistently larger for monotherapy on all clinical and neuroimaging outcomes.

The results we now report demonstrate that there are two classes of effect produced by LMTM treatment in wild-type and tau transgenic mice: those that are subject to dynamic modulation by prior exposure to cholinesterase inhibitor and those which are not. In tau transgenic mice, the treatment effects that can be modulated include increase in ACh release in the hippocampus, changes in synaptic proteins, increase in mitochondrial complex IV activity and reversal of behavioural impairment. The only treatment effects that are not subject to pharmacological modulation are the primary effect on tau aggregation pathology and its immediate effect on neuronal function, as measured for example by restoration of choline acetyltransferase expression in the basal forebrain.

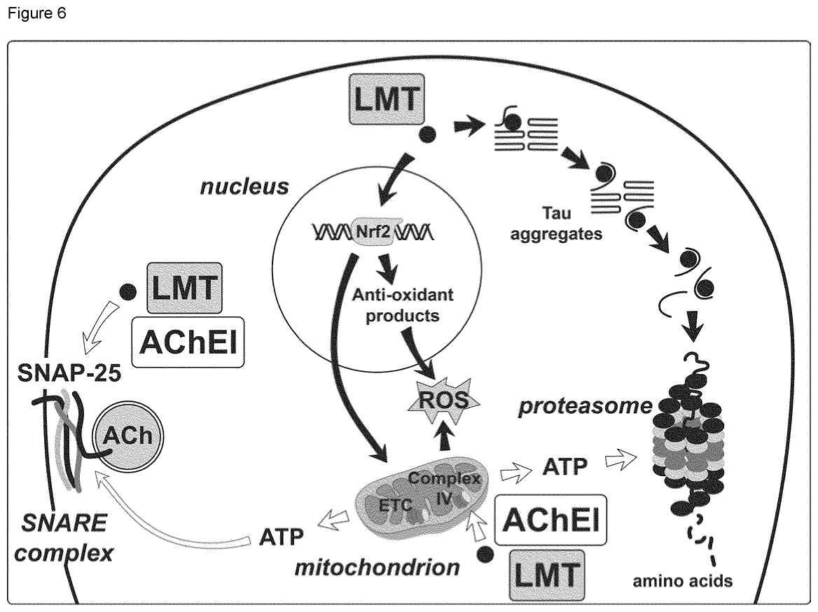

The two classes of LMTM treatment effect are summarised in FIG. 6.

Effects that are subject to pharmacological modulation are themselves of two types: those which are augmented by the effect on tau aggregation pathology and those which are also seen in wild-type mice. Of the outcomes we have measured, positive treatment effects of LMTM given alone in wild-type mice included an increase in ACh levels in hippocampus, and an increase in synaptophysin levels in multiple brain regions. Therefore, LMTM treatment is able to activate neuronal function at therapeutically relevant doses in wild-type mice lacking tau aggregation pathology.

An increase in synaptophysin signals an increase in number or size of the synaptic vesicles that are required for release of neurotransmitters from the presynapse following activation via an action potential. Therefore, an increase in synaptophysin levels appears to be associated with an increase in a number of neurotransmitters needed to support cognitive and other mental functions.

Although it has been reported that the MT moiety is a weak cholinesterase inhibitor (Pfaffendorf et al., 1997; Deiana et al., 2009), this is unlikely to be the mechanism responsible for the increase in ACh levels.

Specifically, further experiments using scopolamine to increase ACh levels (by blocking M2/M4 negative feedback receptors) showed that the increase produced by LMTM was less than that seen with rivastigmine alone, and that the combination was again inhibitory in wild type mice. Under the condition of cholinesterase inhibition used in these experiments (a very small amount of a cholinesterase inhibitor, 100 nanomolar rivastigmine, added to the perfusion fluid), ACh levels in the hippocampus rise, and when they rise strongly enough, they limit additional ACh release by activating pre-synaptic muscarinic receptors of the M2/M4 subtype (so-called negative feedback receptors).

In this situation, adding scopolamine (1 μM) to the perfusion fluid blocks these presynaptic receptors and, as a consequence, ACh levels rise by 3-5 fold. The fact that LMTM is not additive with rivastigmine in these experiments supports the conclusion that LMTM has a different mechanism of action from rivastigmine. In other words, although LMTM has been described as being a weak inhibitor of cholinesterases in high concentrations, the present effects seem to be unrelated to cholinesterase inhibition, because there is no additive effect with small quantities of rivastigmine.

The increase in ACh and synaptophysin levels might theoretically be explained by an increase in presynaptic mitochondrial activity, since the MT moiety is known to enhance mitochondrial complex IV activity (Atamna et al., 2012), and mitochondria have an important role in homeostatic regulation of presynaptic function (Devine and Kittler, 2018). In particular, The MT moiety is thought to enhance oxidative phosphorylation by acting as an electron shuttle between complex I and complex IV (Atamna et al., 2012). The MT moiety has a redox potential of approximately 0 mV, midway between the redox potential of complex I (−0.4 mV) and complex IV (+0.4 mV).

However, direct measurement of complex IV activity in wild type mice did not show any increase following LMTM treatment. The activating effects of LMTM were also not associated with improvement in spatial recognition memory in wild-type mice.

Chronic pretreatment with rivastagmine suppressed the cholinergic activation in the hippocampus and reduced synaptophysin levels more generally in the brain in wild-type mice. This effect is clearly not dependent on the effects of LMTM on tau aggregation pathology, since there is no pathology in wild-type mice. Rather, they point to a generalised homeostatic downregulation that counteracts the effect of combining two drugs which each have activating effects on neuronal function. Presumably, the primary mechanism that would normally protect against excessive levels of ACh in the synaptic cleft would be an increase in AChE activity. Since rivastigmine produces chronic impairment of this control system, pathways that would otherwise be activated by LMTM are suppressed in order to preserve homeostasis in cholinergic and other neuronal systems. Thus, LMTM-induced effects are subject to dynamic downregulation if the brain is already subject to chronic stimulation by a cholinesterase inhibitor.

Although qualitatively similar, the effects of LMTM given alone are much more prominent and more broad-ranging in tau transgenic L1 mice. The most likely explanation for this is that LMTM combines an inhibitory effect on tau oligomers together with inherent activating effects which are not tau-dependent. The reduction in tau oligomer levels following LMTM treatment facilitates a more pronounced activation of synaptic function and release of neurotransmitters such as ACh and glutamate. Likewise, LMTM reverses the spatial memory deficit seen in tau transgenic L1 mice (Melis et al., 2015a). The negative effects seen when LMTM is introduced on a chronic rivastigmine background appears simply to reflect the reversal of the activation seen with LMTM alone.

A deleterious effect of tau oligomers on functioning of synaptic proteins is readily understandable as being the result of direct interference with docking of synaptic vesicles, membrane fusion and release of neurotransmitter. In tau transgenic L1 mice, synaptic vesicular protein levels are no longer linked quantitatively to either the proteins of the SNARE complex or α-synuclein, implying a loss of functional integration between vesicular and membrane-docking proteins at the synapse. The consequence of this can be seen directly as an impairment in glutamate release from synaptosomal preparations from tau transgenic mice, and a restoration of normal glutamate release following treatment with LMTM.

The mechanisms responsible for the mitochondrial effects of LMTM are more complex. The MT moiety is thought to enhance oxidative phosphorylation by acting as an electron shuttle between complex I and complex IV (Atamna et al., 2012). The MT moiety has a redox potential of approximately 0 mV, midway between the redox potential of complex I (−0.4 mV) and complex IV (+0.4 mV). However, LMTM has no effect on complex IV activity in brain mitochondria isolated from wild-type mice. By contrast, a strong effect was seen in tau transgenic L1 mice. This suggests that tau oligomers interfere with mitochondrial metabolism. It has been shown recently that C-terminally truncated tau protein is bound both to the mitochondrial outer membrane and also enters the intermembrane space of mitochondria (Cieri et al., 2018). Truncated PHF-tau protein isolated from brain tissues of AD patients forms SDS-resistant complexes with the voltage-dependent anion-selective channel protein (VDAC; formerly porin) in the mitochondrial outer membrane, and also with ATP synthase subunit 9 and core protein 2 of complex Ill in the intermembrane space (Wischik et al., 1997). These binding interactions are likely to be deleterious to the functioning of the electron transport chain in mitochondria and the effect of LMTM in reducing tau oligomer accumulation in and around mitochondria may contribute to the activation of complex IV seen in L1 mice.

It is not known how homeostatic downregulation resulting from rivastigmine treatment might affect mitochondrial function. Mitochondria are known to be important homeostatic regulators of synaptic function via buffering of Ca²⁺ levels and ATP generation (Devine and Kittler, 2018).

It is striking that the positive effects of LMTM and their reversal or suppression by pretreatment with anticholinesterase can be seen across different transmitter systems and cellular compartments at multiple levels of brain function. This implies that there is no single locus responsible for the interference in the LMTM treatment response. Rather, the negative interaction appears to be part of a generalised homeostatic downregulation in multiple neuronal systems that compensate for the chronic pharmacological activation resulting from blockade of acetylcholinesterase.

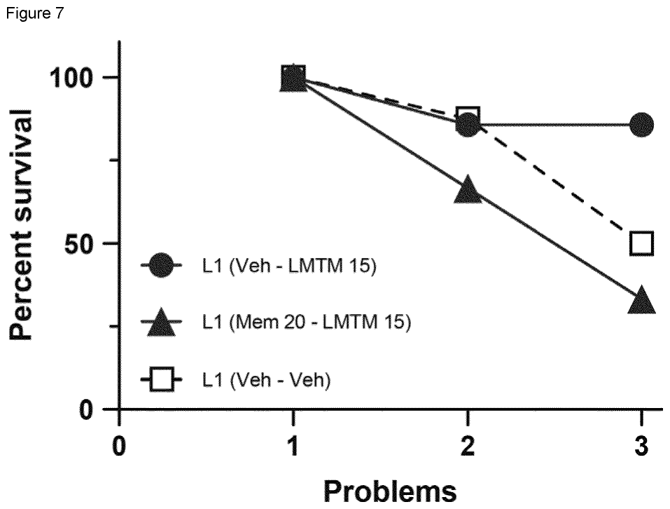

The results with memantine are shown in FIG. 7, and show a similar picture to pretreatment with anticholinesterase. This is as expected, given that the interference in LMTM efficacy seen clinically is very similar for the two drug classes.

More generally, it would be unlikely that the interference affecting LMTM treatment is specific to LMTM. Any treatment that has an activating effect on synaptic function, whether by reducing primary pathology or by another mechanism, is likely to be subject to similar interference, since it is driven primarily by the pre-existing symptomatic treatment.

Thus if clearance of amyloid aggregates results in synaptic activation, as has been proposed Marsh, J, Alifragis, P (2018), then it can be inferred that symptomatic treatments would also interfere with the ability to demonstrate this effect clinically.

A further consideration is whether the homeostatic downregulation that we have demonstrated would operate in the same way if LMTM treatment were primary and symptomatic treatment were added at a later date. The experiments we have conducted to date were originally designed to mimic the clinical situation in which LMTM is added in patients already receiving symptomatic treatments. If homeostatic downregulation is determined by the treatment that comes first, it is logical that the treatment effects of LMTM would dominate, albeit that the response to add-on symptomatic treatment could be reduced to some extent.

In summary, our findings point to the powerful role of homeostatic control systems in the brain. Such systems are well understood and well documented in many neurophysiological contexts. It is therefore entirely plausible that treatment interventions designed to boost neuronal function induce homeostatic controls that limit the extent of neuronal over-activation. In the case of cholinergic function, excessive activity is highly deleterious and results clinically in convulsions, coma and death. This is entirely consistent with chronic stimulation of the brain by symptomatic treatments altering the way in which it responds to other therapeutic interventions.

### Example 4—Withdrawal Analysis for LMTM 8 mg/Day as Monotherapy or Add-On Therapy

Following 18 months of treatment with 8 mg/day LMTM, subjects were required to have a 1 month “washout” followed by cognitive assessment of change. The group was split according to whether they has received LMTM as an add-on to existing symptomatic therapy (AChEI, Memantine, collectively abbreviated to “Achmem”) or as a monotherapy.

The group was further analysed in terms of subjects who had received a high Cmax exposure or a low one. As explained in WO2020/020751, using pharmacokinetic modelling, the 8 mg/day treated population can be split into a group of individuals with “higher” estimated Cmax and a group of individuals with “lower” estimated Cmax. WO2020/020751 explains that splitting of patients according to the threshold of 0.37 ng/ml (that encompasses the 35% of patients with the lowest values) the treatment difference in “high” and “low” Cmax patients receiving the 8 mg/day dose is −3.4 ADAS-cog units.

The numbers in the present analysis were as follows:

| Cmax (exposure) | Add-on to Achmem | Monotherapy |
|---|---|---|
| Low | 120 | 24 |
| High | 242 | 47 |

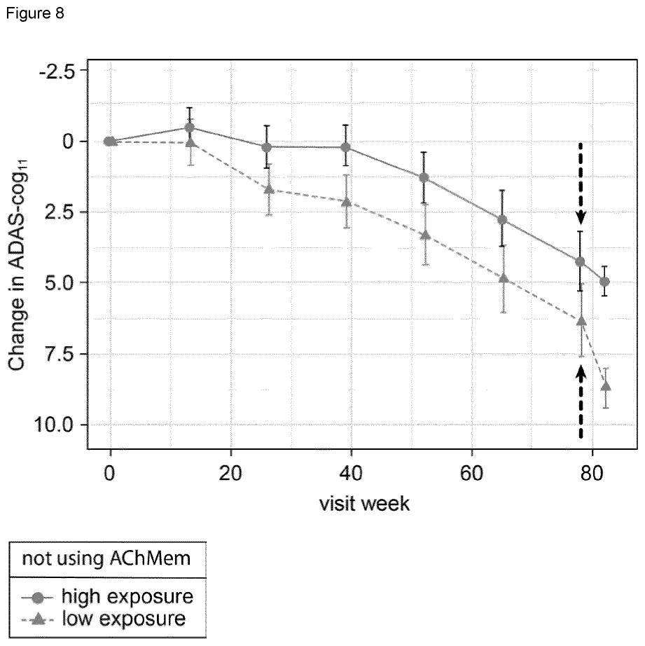

As shown in FIG. 8 (monotherapy) the high Cmax group continue to decline at same rate following LMTM withdrawal supporting a persistent disease-modifying change in rate of cognitive decline following the LMTX treatment period.

The subjects exposed to a lower Cmax suffered a large decline implying that the LMTX benefit in that group may be at least partly symptomatic, and hence not persistent.

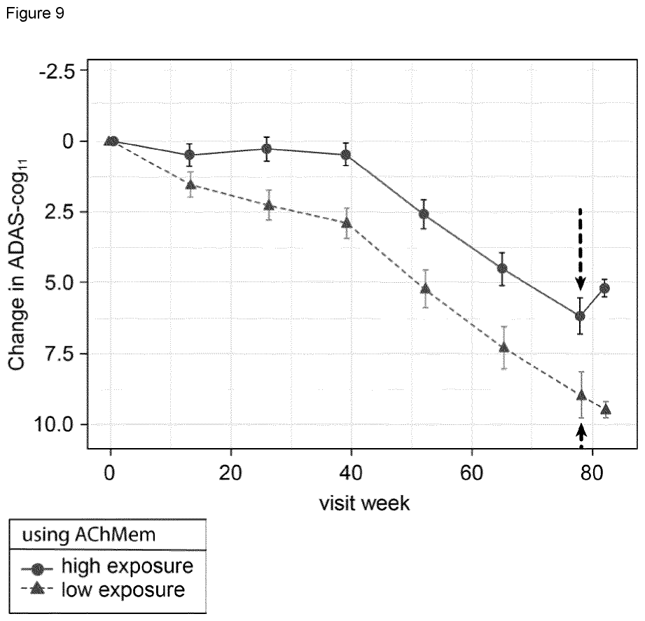

As shown in FIG. 9 (add-on) the high Cmax group actually showed an unexpected improvement following LMTM withdrawal. This is consistent with either a disease-modifying effect during LMTM treatment which results in an improved response to symptomatic treatment alone, or with a negative effect on the symptomatic treatment exhibited by LMTM during the LMTX treatment period.

This finding has potential implications for the use of LMTM and Achmem combination therapies—for example in patient groups who have proved non-responsive to Achmem, the disease modifying effects of LMTX may actually enhance the response to Achmem, particularly once the LMTX is discontinued, and particularly in relation to the ADAS-cog in mild AD subjects.

By contrast the subjects exposed to a lower exposure continued to decline at same rate following withdrawal.

These results support the concentration-response analysis in WO2020/020751. LMTM as monotherapy has significant pharmacological activity at both high and low levels of exposure, but below around 0.38 ng/ml the effects may be more symptomatic than disease-modifying. As an add-on therapy, LMTM has significant pharmacological activity even at low levels of exposure, but an exposure to below ˜0.378 ng/ml as add-on does not give a discernible treatment effect.

### REFERENCES

Congdon, E E, Sigurdsson, E M (2018) Tau-targeting therapies for Alzheimer disease. Nat Rev Neurol 14:399-415
Jaber, V R, Zhao, Y, Sharfman, N M, Li, W, Lukiw, W J (2019) Addressing Alzheimer's disease (AD) neuropathology using anti-microRNA (AM) strategies. Molecular Neurobiology. https://doi.org/10.1007/s12035-019-1632-0.
Kazim, S F, Iqbal, K. 2017. Chapter 2—Neural Regeneration as a Disease-Modifying Therapeutic Strategy for Alzheimer's Disease. In Neuroprotection in Alzheimer's Disease [Ed. Gozes, I.]. pp. 3-29 Academic Press
Panza, F, Lozupone, M, Logroscino, G, Imbimbo, B P (2019) A critical appraisal of amyloid-β-targeting therapies for Alzheimer disease. Nat Rev Neurol 15:73-88
Ustyugov, A, Shevtsova, E, Ashraf, G M, Tarasov, V V, Bachurin, S O, Aliev, G (2018) New therapeutic property of Dimebon as a neuroprotective agent. Current Medicinal Chemistry 25:5315-5326
Al-Hilaly, Y. K., Pollack, S. J., Rickard, J. E., Simpson, M., Raulin, A.-C., Baddeley, T., et al. (2018). Cysteine-independent inhibition of Alzheimer's disease-like paired helical filament assembly by leuco-methylthioninium (LMT). J. Mol. Biol. 430, 4119-4131. doi: 10.1016/j.jmb.2018.08.010.
Atamna, H., Mackey, J., and Dhahbi, J. M. (2012). Mitochondrial pharmacology: electron transport chain bypass as strategies to treat mitochondrial dysfunction. Biofactors 38, 158-166. doi: 10.1002/biof.197.
Baddeley, T. C., Mccaffrey, J., Storey, J. M. D., Cheung, J. K. S., Melis, V., Horsley, D., et al. (2015). Complex disposition of methylthioninium redox forms determines efficacy in tau aggregation inhibitor therapy for Alzheimer's disease. J. Pharmacol. Exptl. Therapeutics 352, 110-118. doi: 10.1124/jpet.114.219352.
Callaway, N. L., Riha, P. D., Bruchey, A. K., Munshi, Z., and Gonzalez-Lima, F. (2004). Methylene blue improves brain oxidative metabolism and memory retention in rats. Pharmacol. Biochem. Behav. 77, 175-181.
Callaway, N. L., Riha, P. D., Wrubel, K. M., Mccollum, D., and Gonzalez-Lima, F. (2002). Methylene blue restores spatial memory retention impaired by an inhibitor of cytochrome oxidase in rats. Neurosci. Lett. 332, 83-86.
Deiana, S., Harrington, C. R., Wischik, C. M., and Riedel, G. (2009). Methylthioninium chloride reverses cognitive deficits induced by scopolamine: comparison with rivastigmine. Psychopharmacology 202, 53-65. doi: 10.1007/s00213-008-1394-2.
Devine, M. J., and Kittler, J. T. (2018). Mitochondria at the neuronal presynapse in health and disease. Nat. Rev. Neurosci. 19, 63-80. doi: 10.1038/nrn.2017.170.
Fitzpatrick, A. W. P., Falcon, B., He, S., Murzin, A. G., Murshudov, G., Garringer, H. J., et al. (2017). Cryo-EM structures of tau filaments from Alzheimer's disease. Nature 547, 185-190. doi: 10.1038/nature23002.
Gauthier, S., Feldman, H. H., Schneider, L. S., Wilcock, G. K., Frisoni, G. B., Hardlund, J. H., et al. (2016). Efficacy and safety of tau-aggregation inhibitor therapy in patients with mild or moderate Alzheimer's disease: a randomised, controlled, double-blind, parallel-arm, phase 3 trial. Lancet 388, 2873-2884. doi: 10.1016/S0140-6736(16)31275-2.
Gonzalez-Lima, F., and Bruchey, A. K. (2004). Extinction memory by the metabolic enhancer improvement methylene blue. Learning & Memory 11, 633-640.
Harrington, C. R., Storey, J. M. D., Clunas, S., Harrington, K. A., Horsley, D., Ishaq, A., et al. (2015). Cellular models of aggregation-dependent template-directed proteolysis to characterize tau aggregation inhibitors for treatment of Alzheimer's disease. J. Biol. Chem. 290, 10862-10875. doi: 10.1074/jbc.M114.616029.
Husain, M., and Mehta, M. A. (2011) Cognitive enhancement by drugs in health and disease. Trends Cognitive Sci. 15, 28-36.
Li, Y. and Kavalali, E. T. (2017). Synaptic vesicle-recycling machinery components as potential therapeutic targets. Pharmacol. Rev. 69, 141-160.
Maier, L. J., Ferris, J. A., and Winstock, A. R. (2018). Pharmacological cognitive enhancement among non-ADHD individuals—A cross-sectional study in 15 countries.” Int. J. Drug Policy 58, 104-112.
Martinez, J. L., Jr., Jensen, R. A., Vasquez, B. J., Mcguiness, T., and Mcgaugh, J. L. (1978). Methylene blue alters retention of inhibitory avoidance responses. Physiological Psychology 6, 387-390.
Marsh, J, Alifragis, P (2018) Synaptic dysfunction in Alzheimer's disease: the effects of amyloid beta on synaptic vesicle dynamics as a novel target for therapeutic intervention. Neural Regen. Res. 13:616-623.
Melis, V., Magbagbeolu, M., Rickard, J. E., Horsley, D., Davidson, K., Harrington, K. A., et al. (2015a). Effects of oxidized and reduced forms of methylthioninium in two transgenic mouse tauopathy models. Behav. Pharmacol. 26, 353-368. doi: 10.1097/fbp.0000000000000133.
Melis, V., Zabke, C., Stamer, K., Magbagbeolu, M., Schwab, K., Marschall, P., et al. (2015b). Different pathways of molecular pathophysiology underlie cognitive and motor tauopathy phenotypes in transgenic models for Alzheimer's disease and frontotemporal lobar degeneration. Cell. Mol. Life Sci. 72, 2199-2222. doi: 10.1007/s00018-014-1804-z.
Mesulam, M. M. (2013). Cholinergic circuitry of the human nucleus basalis and its fate in Alzheimer's disease. J. Comp. Neurol. 521, 4124-4144. doi: 10.1002/cne.23415.
Pepeu, G., and Grazia Giovannini, M. (2017). The fate of the brain cholinergic neurons in neurodegenerative diseases. Brain Res. 1670, 173-184. doi: 10.1016/j.brainres.2017.06.023.
Pfaffendorf, M., Bruning, T. A., Batink, H. D., and Van Zwieten, P. A. (1997). The interaction between methylene blue and the cholinergic system. Br. J. Pharmacol. 122, 95-98. doi: 10.1038/sj.bjp.0701355.
Revett, T. J., Baker, G. B., Jhamandas, J., and Kar, S. (2013). Glutamate system, amyloid beta peptides and tau protein: functional interrelationships and relevance to Alzheimer disease pathology. J. Psychiat. Neurosci. 38, 6-23. doi: 10.1503/jpn.110190.
Riha, P. D., Bruchey, A. K., Echevarria, D. J., and Gonzalez-Lima, F. (2005). Memory facilitation by methylene blue: Dose-dependent effect on behavior and brain oxygen consumption. Eur. J. Pharmacol. 511, 151-158.
Wilcock, G. K., Gauthier, S., Frisoni, G. B., Jia, J., Hardlund, J. H., Moebius, H. J., et al. (2018). Potential of low dose leuco-methylthioninium bis(hydromethanesulphonate) (LMTM) monotherapy for treatment of mild Alzheimer's disease: cohort analysis as modified primary outcome in a phase 3 clinical trial. J. Alzheimer's Dis. 61, 635-657. doi: 10.3233/JAD-170560.
Wischik, C. M., Edwards, P. C., Lai, R. Y. K., Roth, M., and Harrington, C. R. (1996). Selective inhibition of Alzheimer disease-like tau aggregation by phenothiazines. Proc. Natl. Acad. Sci. U.S.A. 93, 11213-11218. doi: 10.1073/pnas.93.20.11213.
Wischik, C. M., Novak, M., Edwards, P. C., Klug, A., Tichelaar, W., and Crowther, R. A. (1988a). Structural characterization of the core of the paired helical filament of Alzheimer disease. Proc. Natl. Acad. Sci. U.S.A. 85, 4884-4888. doi: 10.1073/pnas.85.13.4884.
Wischik, C. M., Novak, M., Thøgersen, H. C., Edwards, P. C., Runswick, M. J., Jakes, R., et al. (1988b). Isolation of a fragment of tau derived from the core of the paired helical filament of Alzheimer disease. Proc. Natl. Acad. Sci. U.S.A. 85, 4506-4510. doi: 10.1073/pnas.85.12.4506.
Wischik, C. M., Schelter, B. O., Wischik, D. J., Storey, J. M. D., and Harrington, C. R. (2018). Modeling prion-like processing of tau protein in Alzheimer's disease for pharmaceutical development. J. Alzheimer's Dis. 62, 1287-1303. doi: 10.3233/JAD-170727.
Wrubel, K. M., Barrett, D., Shumake, J., Johnson, S. E., and Gonzalez-Lima, F. (2007). Methylene blue facilitates the extinction of fear in an animal model of susceptibility to learned helplessness. Neurobiol. Learning Memory 87, 209-217.
Zoellner, L. A., Telch, M., Foa, E. B., Farach, F. J., Mclean, C. P., Gallop, R., et al. (2017). Enhancing extinction learning in posttraumatic stress disorder wWith brief daily imaginal exposure and methylene blue: a randomized controlled trial. J. Clin. Psychiat. 78, e782-e789. doi: 10.4088/JCP.16m10936.

1. A method of therapeutic treatment of Alzheimer's disease or Mild Cognitive Impairment in a subject,
which method comprises administering to said subject a therapeutic methylthioninium (MT)-containing compound,
wherein the MT-containing compound is an LMTX compound of the following formula: 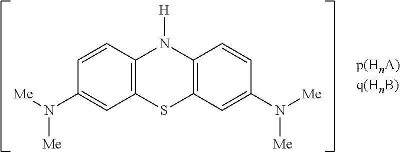
wherein each of HₙA and HₙB (where present) are protic acids which may be the same or different,
and wherein p=1 or 2; q=0 or 1; n=1 or 2; and (p+q)×n=2,
or a hydrate or solvate thereof,
wherein said subject is selected from the following groups:
(i) a subject who has not historically received treatment with a neurotransmission modifying compound which is a modifier of the activity of acetylcholine or glutamate neurotransmitters; or
(ii) a subject who has historically received treatment with a neurotransmission modifying compound which is a modifier of the activity of acetylcholine or glutamate neurotransmitters, but ceased that treatment at least 3, 4, 5, 6, 7, or 8 weeks prior to treatment with the LMTX compound;
wherein said therapeutic treatment with the LMTX compound is maintained for treatment timeframe without co-administration with a neurotransmission modifying compound which is a modifier of the activity of acetylcholine or glutamate neurotransmitters,
followed by;
treatment with the LMTX compound with co-administration of a neurotransmission modifying compound which is a modifier of the activity of acetylcholine or glutamate neurotransmitters.
wherein the treatment timeframe prior to co-administration of the LMTX compound with the neurotransmission modifying compound is at least 2 months.

2. The method as claimed in claim 1 wherein said therapeutic treatment with the LMTX compound comprises a total daily dose of between 2 and 100 mg of MT to the subject per day, optionally split into 2 or more doses.

3. The method as claimed in claim 2 wherein the total daily dose of MT is from 10-60 mg.

4. (canceled)

5. The method as claimed in claim 2 wherein the total daily dose is between 20 and 40 mg.

6. The method as claimed in claim 1 wherein the LMTX compound has the following formula, where HA and HB are different mono-protic acids:

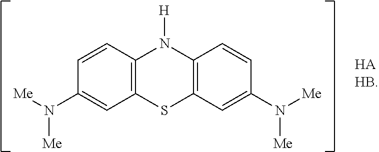

7. The method as claimed in claim 1 wherein the LMTX compound has the following formula: 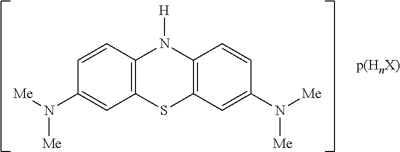
wherein each of HₙX is a protic acid.

8. The method as claimed in claim 1 wherein the LMTX compound has the following formula and H₂A is a di-protic acid:

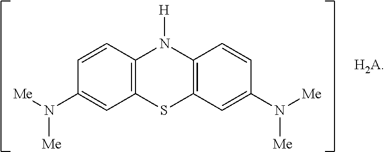

9. The method as claimed in claim 7 wherein the LMTX compound has the following formula and is a bis-monoprotic acid salt:

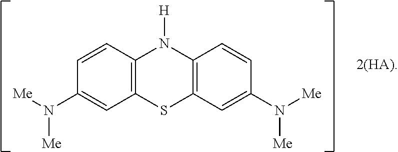

10. The method as claimed in claim 1 wherein the or each protic acid is an inorganic acid.

11. The method as claimed in claim 10 wherein each protic acid is a hydrohalide acid.

12. (canceled)

13. The method as claimed in claim 1 wherein the or each protic acid is an organic acid.

14. The method as claimed in claim 13 wherein the or each protic acid is selected from H₂CO₃, CH₃COOH, methanesulfonic acid, 1,2-ethanedisulfonic acid, ethanesulfonic acid, naphthalenedisulfonic acid, and p-toluenesulfonic acid.

15. The method as claimed in claim 1 wherein the LMTX compound is LMTM:

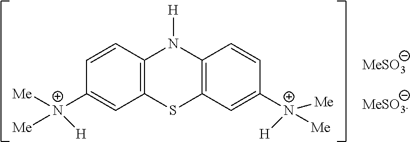

16-17. (canceled)

18. The method as claimed in claim 1 wherein the LMTX compound is selected from the group consisting of:

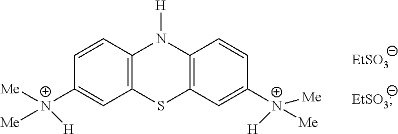 [SMILES: C[NH+](C)c1ccc2c(c1)Sc1cc([NH+](C)C)ccc1N2 ; "EtSO3EtSO3;"]

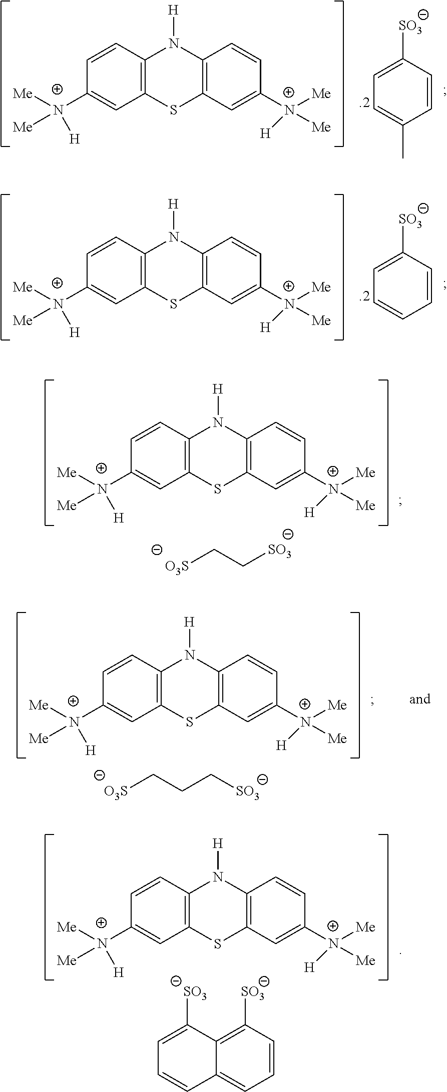

19-29. (canceled)

30. The method as claimed in claim 1 wherein:
(i) the treatment timeframe prior to co-administration of the LMTX compound with the neurotransmission modifying compound is at least 6 months, and/or
(ii) the treatment prior to co-administration of the LMTX compound with the neurotransmission modifying compound is a monotherapy, and/or
(iii) the subject who has historically received treatment with a neurotransmission modifying compound which is a modifier of the activity of acetylcholine or glutamate neurotransmitters has ceased that treatment at least 6 weeks prior to administration of the LMTX compound, or assessment.

31-36. (canceled)

37. A method of therapeutic treatment of Alzheimer's disease or Mild Cognitive Impairment in a subject,
which subject has been selected as being non-responsive to treatment with a neurotransmission modifying compound which is a modifier of the activity of acetylcholine or glutamate neurotransmitters,
which method comprises administering to said subject a therapeutic methylthioninium (MT)-containing compound,
wherein the MT-containing compound is an LMTX compound of the following formula: 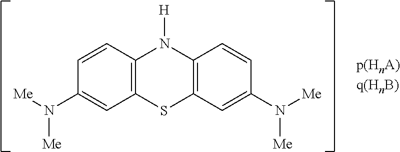
wherein each of HₙA and HₙB (where present) are protic acids which may be the same or different,
and wherein p=1 or 2; q=0 or 1; n=1 or 2; and (p+q)×n=2,
or a hydrate or solvate thereof,
followed by treatment with said neurotransmission modifying compound.

38. The method as claimed in claim 37, wherein the Alzheimer's disease is mild Alzheimer's disease.

39. The method as claimed in claim 37 wherein said therapeutic treatment with the LMTX compound is maintained for treatment timeframe with co-administration with the neurotransmission modifying compound,
followed by;
treatment with the neurotransmission modifying compound without the LMTX compound, wherein optionally the treatment timeframe with co-administration is at least 2 months.

40-41. (canceled)

42. The method as claimed in claim 1, wherein the neurotransmission modifying compound is an acetylcholinesterase inhibitor.

43. The method as claimed in claim 42, wherein the neurotransmission modifying compound is selected from donepezil; rivastigmine; and galantamine.

44. The method as claimed claim 1, wherein the neurotransmission modifying compound is an N-methyl-D-aspartate receptor (NMDA) receptor antagonist.

45. The method as claimed in claim 44, wherein the neurotransmission modifying compound is memantine.

46-47. (canceled)

48. The method as claimed in claim 37 wherein the LMTX compound has the following formula, where HA and HB are different mono-protic acids:

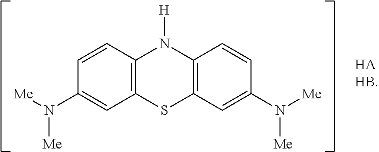

49. The method as claimed in claim 37 wherein the LMTX compound has the following formula: 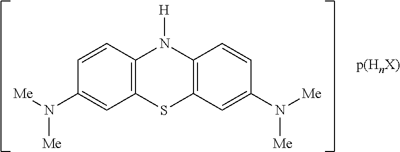
wherein each of HₙX is a protic acid.

50. The method as claimed in claim 37 wherein the LMTX compound has the following formula and H₂A is a di-protic acid:

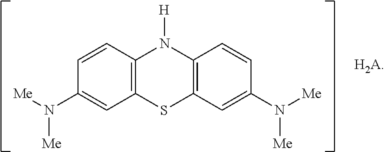

51. The method as claimed in claim 49 wherein the LMTX compound has the following formula and is a bis-monoprotic acid salt:

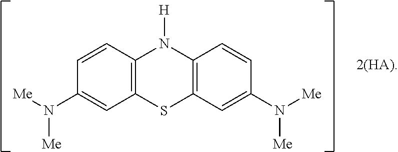

52. The method as claimed in claim 37 wherein the or each protic acid is an inorganic acid, which is optionally a hydrohalide acid.

53. The method as claimed in claim 52, wherein the or each inorganic acid is a hydrohalide acid.

54. The method as claimed in claim 37 wherein the or each protic acid is an organic acid.

55. The method as claimed in claim 54, wherein the or each organic acid is selected from H₂CO₃, CH₃COOH, methanesulfonic acid, 1,2-ethanedisulfonic acid, ethanesulfonic acid, naphthalenedisulfonic acid, p-toluenesulfonic acid.

56. The method as claimed in claim 37 wherein the LMTX compound is LMTM:

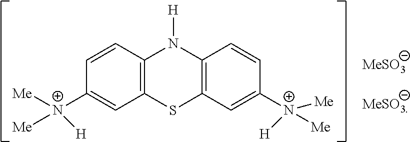

57. The method as claimed in claim 37 wherein the LMTX compound is selected from the group consisting of: [chemical structure]
